SEQUENCE LISTING (54 sequences)

SEQ ID NO: 1 - DNA, 35 bp, Artificial Sequence
  Description of Artificial Sequence Synthetic primer
aaggatccatggagggttcgtccaaagggctgcga

SEQ ID NO: 2 - DNA, 35 bp, Artificial Sequence
  Description of Artificial Sequence Synthetic primer
aacctaggctaatcaaatttcacagtctctccatc

SEQ ID NO: 3 - DNA, 34 bp, Artificial Sequence
  Description of Artificial Sequence Synthetic primer
aaggatccatggagggttcgtccaaagggtgagg

SEQ ID NO: 4 - DNA, 38 bp, Artificial Sequence
  Description of Artificial Sequence Synthetic primer
aacctaggcagactccaaagttgctcaacgtcaaacgc

SEQ ID NO: 5 - DNA, 25 bp, Artificial Sequence
  Description of Artificial Sequence Synthetic primer
atggtgatggctggtgcttcttctt

SEQ ID NO: 6 - DNA, 24 bp, Artificial Sequence
  Description of Artificial Sequence Synthetic primer
ttagagaggaacgctgtgcaagac

SEQ ID NO: 7 - DNA, 25 bp, Artificial Sequence
  Description of Artificial Sequence Synthetic primer
ggtgatatatagccataattgtgtt

SEQ ID NO: 8 - DNA, 25 bp, Artificial Sequence
  Description of Artificial Sequence Synthetic primer
ggtatatatagctcctgcaattata

SEQ ID NO: 9 - DNA, 31 bp, Artificial Sequence
  Description of Artificial Sequence Synthetic primer
gatatcaagaggataagaggattgtgttgga

SEQ ID NO: 10 - DNA, 31 bp, Artificial Sequence
  Description of Artificial Sequence Synthetic primer
agatctcttaaatgatctcccacacctcaaa

SEQ ID NO: 11 - DNA, 31 bp, Artificial Sequence
  Description of Artificial Sequence Synthetic primer
gatatcaacttttttgcttctcctttttcaa

SEQ ID NO: 12 - DNA, 31 bp, Artificial Sequence
  Description of Artificial Sequence Synthetic primer
agatctttttcactgcttgttttggtatcta

SEQ ID NO: 13 - DNA, 31 bp, Artificial Sequence
  Description of Artificial Sequence Synthetic primer
gttaacgattcgactcggtcacgtgatatac

SEQ ID NO: 14 - DNA, 31 bp, Artificial Sequence
  Description of Artificial Sequence Synthetic primer
agatctgtcagagtttgactatggagcaaac

SEQ ID NO: 15 - DNA, 31 bp, Artificial Sequence
  Description of Artificial Sequence Synthetic primer
gatatcataattatgtcaatcttgcgtgttt

SEQ ID NO: 16 - DNA, 31 bp, Artificial Sequence
  Description of Artificial Sequence Synthetic primer
agatcttttctcttcaaaatccaaaacagag

SEQ ID NO: 17 - DNA, 31 bp, Artificial Sequence
  Description of Artificial Sequence Synthetic primer
gatatctaggccttgtaatatttttgatgaa

SEQ ID NO: 18 - DNA, 31 bp, Artificial Sequence; identical to SEQ ID NO: 12
  Description of Artificial Sequence Synthetic primer

SEQ ID NO: 19 - DNA, 31 bp, Artificial Sequence
  Description of Artificial Sequence Synthetic primer
gttaacatggcgctcttgtctcaagcaggag

SEQ ID NO: 20 - DNA, 31 bp, Artificial Sequence
  Description of Artificial Sequence Synthetic primer
gttaacttatagacaccttttgttcacgtcc

SEQ ID NO: 21 - DNA, 31 bp, Artificial Sequence
  Description of Artificial Sequence Synthetic primer
gttaacatggaatcacagaaacccattttcg

SEQ ID NO: 22 - DNA, 31 bp, Artificial Sequence
  Description of Artificial Sequence Synthetic primer
gttaactcaattcagataaatgctgtccaag

SEQ ID NO: 23 - DNA, 29 bp, Artificial Sequence
  Description of Artificial Sequence Synthetic primer
ggatccatggctatggcgagtttatatgc

SEQ ID NO: 24 - DNA, 29 bp, Artificial Sequence
  Description of Artificial Sequence Synthetic primer
gctagcctaataggcaggagcagcgagat

SEQ ID NO: 25 - DNA, 28 bp, Artificial Sequence
  Description of Artificial Sequence Synthetic primer
agatctatggcgctacaggatctggacg

SEQ ID NO: 26 - DNA, 29 bp, Artificial Sequence
  Description of Artificial Sequence Synthetic primer
gctagctcagtcaacatcggaggtagata

SEQ ID NO: 27 - DNA, 29 bp, Artificial Sequence
  Description of Artificial Sequence Synthetic primer
ccatggatggaaaacgacgtcaaagagaa

SEQ ID NO: 28 - DNA, 29 bp, Artificial Sequence
  Description of Artificial Sequence Synthetic primer
gctagcctagctattggagacggacactc

SEQ ID NO: 29 - DNA, 29 bp, Artificial Sequence
  Description of Artificial Sequence Synthetic primer
ggatccatggctatggcgagtttatatcg

SEQ ID NO: 30 - DNA, 29 bp, Artificial Sequence
  Description of Artificial Sequence Synthetic primer
gctagcctaataggcaggagcagcgagat

SEQ ID NO: 31 - DNA, 30 bp, Artificial Sequence
  Description of Artificial Sequence Synthetic primer
gttaacatgtctatggcgagtttgtatcgg

SEQ ID NO: 32 - DNA, 30 bp, Artificial Sequence
  Description of Artificial Sequence Synthetic primer
gttaacttaggcaggagcagagagttcttc

SEQ ID NO: 33 - DNA, 28 bp, Artificial Sequence
  Description of Artificial Sequence Synthetic primer
gatatccttgagtcatacatctatgata

SEQ ID NO: 34 - DNA, 31 bp, Artificial Sequence
  Description of Artificial Sequence Synthetic primer
agatctccatggtttagtgattgaaccggtg

SEQ ID NO: 35 - DNA, 27 bp, Artificial Sequence
  Description of Artificial Sequence Synthetic primer
gatatcgataattctttaatttactgg

SEQ ID NO: 36 - DNA, 28 bp, Artificial Sequence
  Description of Artificial Sequence Synthetic primer
ggatccgctaatatgtgaaaggttgtac

SEQ ID NO: 37 - DNA, 29 bp, Artificial Sequence
  Description of Artificial Sequence Synthetic primer
gttaacccctaatgaccacatcaaccttg

SEQ ID NO: 38 - DNA, 26 bp, Artificial Sequence
  Description of Artificial Sequence Synthetic primer
agatctgatgatggcggaagaaggag

SEQ ID NO: 39 - DNA, 28 bp, Artificial Sequence
  Description of Artificial Sequence Synthetic primer
gatatcccaaagcagcaaccatttttcc

SEQ ID NO: 40 - DNA, 29 bp, Artificial Sequence
  Description of Artificial Sequence Synthetic primer
agatctgtattttaaacgtatcaagttcc

SEQ ID NO: 41 - DNA, 27 bp, Artificial Sequence
  Description of Artificial Sequence Synthetic primer
gttaacatggcgacctccgtcgataac

SEQ ID NO: 42 - DNA, 28 bp, Artificial Sequence
  Description of Artificial Sequence Synthetic primer
gttaacctagaagattaagagatcctcc

SEQ ID NO: 43 - DNA, 28 bp, Artificial Sequence
  Description of Artificial Sequence Synthetic primer
gttaactcggctgacgcgcctcctagtc

SEQ ID NO: 44 - DNA, 28 bp, Artificial Sequence
  Description of Artificial Sequence Synthetic primer
gttaacgaatatcaagaaatcctccttg

SEQ ID NO: 45 - DNA, 31 bp, Artificial Sequence
  Description of Artificial Sequence Synthetic primer
gttaacatgacttctttctctctcactttca

SEQ ID NO: 46 - DNA, 31 bp, Artificial Sequence
  Description of Artificial Sequence Synthetic primer
gttaactcaatcttcattctcttctctttct

SEQ ID NO: 47 - DNA, 31 bp, Artificial Sequence
  Description of Artificial Sequence Synthetic primer
gttaacatgggttctactgatgagcccagaa

SEQ ID NO: 48 - DNA, 31 bp, Artificial Sequence
  Description of Artificial Sequence Synthetic primer
gttaactcaagcattgttggttgcgttccct

SEQ ID NO: 49 - DNA, 31 bp, Artificial Sequence
  Description of Artificial Sequence Synthetic primer
gttaacatgtccgcactgttcgaaccctaca

SEQ ID NO: 50 - DNA, 31 bp, Artificial Sequence
  Description of Artificial Sequence Synthetic primer
gttaactcagcgatagcgctcaagccagtgc

SEQ ID NO: 51 - DNA, 31 bp, Artificial Sequence
  Description of Artificial Sequence Synthetic primer
gttaacatggcaatcattttcgatccgatca

SEQ ID NO: 52 - DNA, 31 bp, Artificial Sequence
  Description of Artificial Sequence Synthetic primer
gttaacttacagcgtcgggtagtcgatgtag

SEQ ID NO: 53 - DNA, 20 bp, Artificial Sequence
  Description of Artificial Sequence Synthetic primer
gttaacatggccgctaaaag

SEQ ID NO: 54 - DNA, 24 bp, Artificial Sequence
  Description of Artificial Sequence Synthetic primer
gttaacgctatcaatgtacaaagc

(19) United States
(12) Patent Application Publication
(10) Pub. No.: US 2005/0289662 A1
(43) Pub. Date: Dec. 29, 2005

(54) Reporter system for plants

(76) Inventor: Carsten Meier, Copenhagen (DK)

Correspondence Address: Gabor L Szekeres;Law Offices of Gabor L Szekeres, 8141 Kaiser Boulevard, Suite 112, Anaheim, CA, 92808, US

(21) Appl. No.: 10/515,988

(22) PCT Filed: May 30, 2003
(86) PCT No.: PCT/IB03/02081

§ 371 Date: May 9, 2005

(30) Foreign Application Priority Data
May 29, 2002 (DK) PA200200823

(51) Int. Cl. A01H 1/00; C12N 15/82

(52) U.S. Cl. 800/278; 435/468

(57) ABSTRACT

A reporter system capable of giving rise to a directly monitorable phenotypic trait in a plant, in the presence of an outer stimulus such as for example a pollutant, is provided. The system optionally also has the ability to remediate soil. Genetically modified plants comprising said reporter system and optionally the remediation capability, a process for detection of soil pollution and optionally for bioremediating soil by employing said genetically modified plants, as well as the use of genetically modified plants for monitoring soil pollution and optionally for bioremediating soil are also provided.

### FIELD OF THE INVENTION

The present invention relates to a reporter system which is capable of giving rise to a directly monitorable phenotypic trait in a plant in the presence of an outer stimulus such as for example a pollutant and optionally also comprises a system which, when present in said plant, may be used to bioremediate soil. The present invention also relates to genetically modified plants comprising said reporter system and optionally also said bio-remediation system, a process for detection of soil pollution and optionally for bioremediating soil by employing said genetically modified plants, as well as the use of genetically modified plants for biodetection of soil pollution and optionally for bioremediating soil.

### BACKGROUND

Soil pollution may cause serious adverse effects on the environment and on human and animal health. The pollution is a consequence of industrial, agricultural and other human activities, and poses a serious and growing problem. In Denmark, for example, the Danish Ministry of Environment estimated that the number of industrially polluted locations in Denmark were 14,000 in 1995 (Miljøtilstandsrapport 1997). The pollution may involve a large number of chemical compounds of both inorganic and organic nature.

Inorganic pollutants can for example be heavy metals. These can be found at various concentrations in different types of soil and can, unlike organic pollutants, not be chemically converted or biodegraded by microorganisms (Zhu et al., 1999). In trace amounts certain heavy metals such as cupper (Cu) and Zinc (Zn) perform vital structural rolls as cofactors in enzyme homeostasis, but when in excess these heavy metals, as well as non-essential metals such as cadmium (Cd), mercury (Hg) and lead (Pb), are toxic. A number of human disorders have been implicated to be connected to the ingestion of heavy metals, e.g. have Cd been shown to increase the rate of cancer.

A large number of organic pollutants are also found in soil. Examples are xenobiotic compounds containing nitro functional groups, which are used in the production of agricultural chemicals, pharmaceuticals, dyes and plastics (Gorontzy et al. 1994, Spain et al. 1995, White & Snape. 1993). Such compounds are also used in mining, farming and they are the main charge In ammunition including land mines. The most common residues contain 2,4,6-trinitrotoluene (TNT), hexahydro-1,3,5-trinitro-1,3,5-triazine (RDX), octahydro-1,3,5,7-tetranitro-1,3,5,7-tetrazocine (HMX), and associated impurities and environmental transformation products. Such compounds contaminate their sites of manufacture and storage as well as military installations (Sheng et al 1998, Taha et al., 1997). In addition, it is estimated that approximately 90% of the mines currently in use are leaking (Boline 1999), resulting in the spread of TNT into the soil. Unlike many other pollutants, some of these contaminants have little affinity for soils and rapidly migrate to pollute groundwater. This is a concern as high levels of TNT have been observed to have the potential to inhibit biological activity (Gong et al., 1999). Besides the direct consequences of the pollution itself, pollutants of this type may be an indication of the presence of explosives. As land mines are killing and maiming people in former war zones, particularly in remote and poor parts of the world, knowledge of their presence would be of great value.

Detection

A first requirement in dealing with soil pollution, is an ability to detect polluted sites. Detection systems that are practical and relatively inexpensive are desirable, in order to facilitate their wide-spread use. The currently available detection methods allow for the detection of pollutants, but the methods are both inconvenient and costly.

When referring to information concerning a soil sample each observation relates to a particular location and time. Knowledge of an attribute value, say a pollutant concentration, is thus of little interest unless location and/or time of measurement are known and accounted for in the analysis. The key decisions to achieve cost-effective, accurate site characterizations are the number, location and type of soil samples to be collected. Site characterization errors occur when the sample does not accurately represent the area which the modeling plan assumes it represents. This is a particular problem when the contaminant is distributed nonhomogeneously throughout the soil, as occurs with e.g. explosives contamination.

Thus, the characterization of contaminated soils can be expensive and time consuming due to the large number of samples required to effectively evaluate a site. Present laboratory methods of evaluating environmental samples offer high sensitivity and the ability to evaluate multiple chemicals, but the time and cost associated with such methods often limit their effectiveness. Thus, for many applications there exists a requirement for an economically feasible, real-time, in-situ system for the mapping of contaminated soils.

Among the techniques presently in use for the detection of heavy metals is in-situ soil contamination sensor In (LIBS) laser induced breakdown spectroscopy (Cremers et al. 2001).

Soil contaminated by explosives are traditionally monitored by collecting samples which are analysed in a laboratory by applying various techniques, such as Enzyme Immunoassay and High Performance Liquid Chromatography (Haas et al. 1995).

The detection of land mines is normally carried out by sweeping the concerned area using metal-detectors, dogs or manual labour. In military demining the objective is to clear a minefield as fast as possibel using brute force, and usually a clearance rate of 80-90% is accepted. Humanitarian demining, on the other hand, is more difficult and dangerous, as it requires the complete removal of all mines and the return of the cleared minefield to normal use. Today, most humanitarian demining is done using handheld metal detectors finding objects containing metal by utilizing a time varing electromagnetic field to induce eddy-currents in the object. Which in turn generates a detectable magnetic field. Old landmines contain metal parts (e.g. the firing pin), but modem landmines contain very small amounts or no metal at all. Increasing the sensitivity the detector to detect smaller amounts of metal also makes it very sensitive to metal scrap often found in areas where mines may be located. Furthermore, metal detectors, however sophisticated can only succeed in finding anomalies in the ground without providing information about whether an explosive agent is present or not. One major problem in humanitarian demining is to discriminate between a “dummy” object and a landmine. Identifying and removing a harmless object is a time-consuming and costly process. Dogs have extremely well-developed olifactory senses and can be trained to detect explosives in trace quantities. This technique, however requires extensive training of the dogs and their handlers, and the dog's limited attention span makes it difficult to maintain continuous operations. A number of mine detection techniques are emerging as complements to presently used methods. They include ground penetrating radar (GPR), infrared thermography and advanced metal detectors. A common feature of these techniques Is that they detect “anomalies” in the ground but are unable to indicate the presence of an explosive agent. Basically, GPR systems work by emitting a short electromagnetic pulse in the ground through a wideband antenna. Reflections from the ground are then measured to form a vector. The displacement of the antenna allows to build an image by displaying successive vectors side by side. High frequencies are needed to achieve a good spatial resolution, but penetration depth of electric fields being inversely proportional to the frequency, too high frequencies are useless after some centimeters. Hence the choice of the frequency range is a tradeoff between resolution and penetration depth (Borgwardt, C. 1995). Although the detectors can be tuned to be sensitive enough to detect the small amount of metal in modern mines, this is not practically feasible, as it will also lead to the detection of smaller debris and augment the false alarms rate. The only current alternative is to prod the soil at a shallow angle using rigid sticks of metal to determine the shape of an object; this is an intrinsically dangerous operation.

Plants have prevoisly been employed as an indication for the presence of analytes in the field. Such use have typically been a crude indication of the presence of analytes based on naturally occuring plant-life, For example have ‘indicator’ plants been used to locate sites with lucrative mining potential for a long time as the presence of metals in the ground have an effect on plant-life. This could provide mining geologists with an idea whether high amounts of certain metals were present in the ground based primarily on the presence/absence of certain naturally occurring species of plants and analysis of the colleted tissue from plant species known to accumulate metals naturally (Raines and Canney 1980). However, the use of indicator plants in the field, which are refined to give a more specific and sensitive response, e.g. in the form of genetically modified plants have not been described.

In the laboratory, reporter systems have been employed for years for detection and possibly quantification of analytes. The construction of such sophisticated laboratory reporter systems normally involves genetic engineering. Genetically modified plant systems have also been utilised to study the expression of both plant genes and genes originating from animals, microorganisms etc., typically by the application of reporter genes. A reporter gene traditionally encodes an enzyme with an easily assayable activity that is used to report on the transcriptional activity of a gene of interest. Using recombinant DNA methods, the original promoter of the reporter gene is removed and replaced by the promoter of the gene to be studied. The new chimeric gene is introduced into an organism and the expression of the gene of interest is monitored by assaying for the reporter gene product. A reporter gene allows for the study of expression of a gene for which the gene product is not known or is not easy to identify. To determine the patterns of expression of environmentally or developmentally regulated genes, reporter genes are placed under the transcriptional regulation of promoters that show interesting developmental and/or stress responses. In bacteria, the lacZ gene encoding β-galactosidase can be used as a reporter in bacteria that are naturally lac-, or that are lac- due to a mutation. This gene can also be used in many animal systems. Other reporter gene systems which are often used in animals and bacteria where no endogeneous gene exist, include cat (encoding the enzyme chloramphenical acetyl transferase), fus (encoding the jellyfish green fluorescent protein), and lux (encoding the enzyme firefly luciferase). As plants contain endogenous lacZ, this is not generally a useful reporter gene for plants. A widely used reporter gene in plants is the uidA, or gusA, gene that encodes the enzyme β-glucuronidase (GUS) (Kertbundit et al., 1991). This enzyme can cleave the chromogenic (color-generating) β-D-glucuronic acid; substrate X-gluc (5-bromo-4-chloro-3-indolyl) resulting in the production of an insoluble blue color in those plant cells displaying GUS activity. Plant cells themselves do not contain any GUS activity, so the production of a blue color when stained with X-gluc in particular cells indicates the activity of the promoter that drives the transcription of the gusA-chimeric gene in that particular cell. Plants carrying such reporter genes could in principle be useful in the detection of soil pollution, but such use has not been described. A possible explanation for this is, that the reporter systems normally require both a large number of samples to be taken as well as an analysis conducted by highly trained personnel involving sophisticated equipment and the use of expensive chemicals. For practical purposes concerning the monitoring of soil pollution, traditional reporter systems are therefore not feasible.

Remediation

Another requirement in dealing with soil pollution is the ability to remove it. This is normally achieved by simply removing the polluted soil or by remediating the soil by either chemical or biological breakdown of the pollutant.

In dealing with inorganic pollutants such as heavy metals, physical removal of the metals is required, because most of these metals cannot be degraded in the soil. Current practical methods used to decontaminate such sites therefore involve physical excavation of topsoils, transport and reburial elsewhere. In addition a number of soil remediation technologies are also available In the market today, but only a few usable for remediation of heavy metals. Some of the more common remediation techniques are; Landfill disposal, chemical or physical fixation and disposal, Electro-reclamation, Bioventing, and soil washing.

Phytoremediation is the use of green plants to remove, contain, or render harmless environmental contaminants such as heavy metals, trace elements, organic compounds, and radioactive compounds. This low-tech, low-cost cleanup technology can be applied to contaminated soils, groundwater, and wastewater. Compared to conventional remediation methods, phytoremediation is cheaper, easier, and more environment-friendly. A tremendous amount of money is necessary to clean up metal-polluted sites by using traditional engineering methods. Furthermore traditional methods destroy the soil structure and leave it biologically inactive. Use of green plants to decontaminate heavy metals in soils, known as phytoremediation, is an emerging technique that offers the benefits of being in situ, low cost and environmentally sustainable. Another advantage of phytoremediation is that, Instead of removing the contaminated soil and replacing it with fill dirt, the cleanup is done without disturbing the site. After the heavy metals accumulate in plant tissue, the shoots can be harvested and burned. If economically feasible, the metals contained in the ash can be recycled. Otherwise, the ash is disposed of in a suitable landfill. The cost associated with phytoremidiation depends on a number of factors including the density of soil, area of site contaminated, transportation and landfill costs. The same equipment is used in phytoremediation as are common in agricultural practices. In some cases, the costs of phytoremediation can be equated to the local costs to plant crops. Phytoremediation also lacks the need for the removal of large masses of soil. In fact, no soil need be removed, just the plants. This decreases the disposal mass from 30,000 tons, for a sample 10 acre site with the extraction method, to less than 5%, or 1400 tons. This results in tremendous savings when compared to the extraction method. A sample 10 acre site may cost between $3.5-4.5 million for the traditional extraction method, where as, the same site would only cost $1.0-1.2 million for phytoremediation. These savings typically average about 75-85% over the cost of the conventional method. In addition to the economic benefits, phytoremediation is less environmentally destructive than the traditional method due to the fact that the soil is not removed and the metals may be reclaimed for the plant residue. Other problems addressed by the use of phytoremidiation includes wastewater treatment plants.

Wastewater treatment plants have problems since a wide variety of toxic pollutants can be present in sanitary wastewater, including heavy metals. Since these heavy metals are neither broken down nor rendered harmless by biological treatment, they also can be released into the receiving lake or sea.

Knowledge of the uptake of metals by plants has existed for quite some time, but application of this knowledge to phytoremediation is relatively new.

Rugh, et al., (1996) describes genetic engineering employed to develop plants that can enhance removal of metal toxicants such as mercury, utilizing bacterial genes inserted into a plant that is normally considered a weed.

WO9922885 concerns a method for remediating soils contaminated with metal ions, comprising utilization of plants of the genus Pelargonium, to hyperaccumulate metal ions in their roots and shoots. This disclosure also mentions the use of Pelargonium sp. transformed with a gene sequence enhancing the plants ability to take up metals, e.g. a recombinant metallothionein gene or phytochelatin gene or a gene that is biologically functionally equivalent to these genes.

Bioremediation is currently being used to manage municipal sewage, clean up oil spills, remediate ground water contaminated by underground storage leaks, treat industrial waste water, and reclaim a variety of hazardous waste sites.

Examples of bioremediation includes sewage sludge which is applied as fertilizers to cultivated land (Hesselsoe et al. 2001). Genetic engineering has allowed for the introduction of microbial enzyme activities to plants. An example of this is Glyphosate or Roundup((R)) which is the most extensively used herbicide for broad-spectrum control of weeds. Glyphosate inhibits 5-enolpyruvylshikimate-3-phosphate synthase (EPSPS), a key enzyme in the aromatic amino acid biosynthetic pathway in microorganisms and plants (He et al. 2001). There are marked differences in the pattern of host gene expression in incompatible plantmicrobial pathogen interactions compared with compatible interactions, associated with the elaboration of inducible defenses. Constitutive expression of genes encoding a chitinase or a ribosome-inactivating protein in transgenic plants confers partial protection against fungal attack (Lamb et al. 1992). Two bacterial antibiotic resistance genes, one coding for the neomydn phosphotransferase (NPT I) from Tn903, and the other coding for the chloramphenicol acetyltransferase from Tn9 were used as plant selectable markers. Both genes were introduced into the Nicotiana tabacum genome in a new plant expression vector (Pietrzak et al. 1986)

However, a prerequisite of applying phytoremediation, for either inorganic or organic pollutants, normally is that the contaminated location is known and that monitoring of the remediation process takes place by applying traditional methods. By applying a combined plant detection- and bioremediation system it will be possible to identify polluted sites and bio-remediate these in one step. Such a combined system has not previously been described.

In view of the above it is an object of the present invention to provide a reporter system which may be applied in plants to detect an analyte such as for example a form of pollution which is present in the soil, said reporter system being:
specific and sensitive
directly monitorable with no requirements for laboratory facilities or laboratory personnel
applicable in the field and thus facilitating the monitoring of large areas avoiding sampling issues
relatively inexpensive

### SUMMARY OF THE INVENTION

The present invention provides a reporter system capable of giving rise to a directly monitorable phenotypic trait in a plant in the presence of an outer stimulus, comprising a gene encoding a product which is involved in the development of said directly monitorable phenotypic trait in response to the presence of said outer stimulus. The present invention furthermore provides a reporter system wherein the directly monitorable phenotypic trait in the plant is a result of altered expression of said gene.

According to one aspect of the invention the outer stimulus is a pollutant present in the soil in which the plant is growing.

According to another aspect of the invention, the reporter system further comprises a soil bioremediation system.

In a further aspect of the invention, plants carrying the reporter system according to the present invention are provided.

In a further aspect of the invention, a process for biodetection is provided comprising the steps of
Introduction of seeds from a plant according to the present invention and
Monitoring the phenotype of the resulting plants, and optionally
Bioremediating the soil by removing the plants if they accumulate the pollutant.

In another aspect of the present invention, is provided the use of plants according to the present invention for the detection of pollutants and optionally for bioremediation.

The invention is described in greater detail hereinafter.

### DETAILED DESCRIPTION OF THE INVENTION

The present invention provides a novel type of reporter system for plants. The essential component of said reporter system is a gene which is not part of the natural plant genome, i.e. a gene of different origin or a gene from the plant genome in which the coding sequence, the copy number, the location(s) in the genome or the expression has been altered from what is found naturally in that plant, and which encodes a product that is involved in the development of a phenotypic trait in the presence of an outer stimulus. It is an essential feature that said phenotypic trait can be monitored directly, i.e. in the field without the need for sampling and performing complex laboratory-type analyses. The reporter system provided by the present invention could in principle be applied also in other organisms than plants such as for example animals, e.g. insects, microorganisms, e.g. bacteria, or fungi.

The reporter system of the present invention may give rise to a phenotypic trait as a result of the presence of the outer stimulus by two principal mechanisms.

The first possibility is that the outer stimulus interacts with a feature originating from the reporter system. This feature originating from the reporter system may also be present when the outer stimulus is absent, in which case the phenotypic trait does not develop. An example of this is a reporter system according to the present invention comprising a e.g. constitutively expressed gene encoding a gene product which, in the presence of the outer stimulus, gives rise to for example a distinct plant colour.

The second possibility is that the outer stimulus may give rise to a phenotypic trait as a result of altered expression of said gene in the presence of an outer stimulus. The phenotypic trait develops as a result of said altered gene expression. The altered gene expression may be a result of altered transcriptional- or translational activity as well as altered stability/halflife of mRNA or gene products and may involve one or more steps. An example of this is a reporter system according to the present invention comprising a gene, the transcription of which is regulated by a promoter which is active only in the presence of the outer stimulus and which encodes a gene product giving rise to for example a distinct plant colour.

Regardless of the mechanism by which the phenotypic trait develops, the outer stimulus may either exert its influence directly, i.e. involving the analyte itself or indirectly by involving for example a breakdown product of the analyte or another entity, the form or concentration of which is dependent on the presence of the analyte.

The examples mentioned above are included for descriptive purposes only and should not limit the scope of protection of the present invention. It will be evident to a person skilled in the art that it will be possible to develop many particular reporter systems based on different mechanisms without deviating from the gist of the present invention. Consequently, such reporter systems are encompassed by the present invention.

In a preferred embodiment of the present invention, a reporter system capable of giving rise to a directly monitorable phenotypic trait in the form of a distinct colour was developed, allowing for visual inspection of plants carrying said reporter system and furthermore comprising promoters induced by specific stimuli, such as, but not limited to, heavy metals or nitro-containing compounds derived from explosives. In this particular preferred embodiment, the combination of the distinct colouration of the plants and said inducible promoters allows for the screening of large areas of soil for the presence of heavy metal contaminations or explosives.

The present invention facilitates, as opposed to persisting methods, the detection of analytes without the use of laboratory assays. A major benefit of the system is that no sampling is necessary, and that the test can be conducted also in remote areas without the laboratory facilities needed for the conventional test methods. The system furthermore does not require the application of an expensive substrate, such as luciferin or X-gluc, in order to obtain a detectable signal. The present invention, thus, offers an inexpensive alternative to the presently employed reporter systems.

It is an aspect of the present invention to provide a reporter system capable of giving rise to a directly monitorable phenotypic trait in a plant in the presence of an outer stimulus, comprising a gene encoding a product which is involved in the development of said directly monitorable phenotypic trait in response to the presence of said outer stimulus.

The term “reporter system” as used throughout this specification and the appended claims shall be taken to mean any system which is able to transform a stimulus into another feature which can be monitored or measured.

The term “directly monitorable phenotypic trait” as used throughout this specification and the appended claims shall be taken to mean any phenotype of physical or chemical nature which may be monitored without the need for sampling. Such a phenotype may e.g. involve, viability, growth rate, size, shape, colour, colour-pattern, odoeur and taste.

The term “outer stimulus” as used throughout this specification and the appended claims shall be taken to mean any stimulus of external origin of chemical or physical nature which affects a plant.

In a further aspect of the present invention said directly monitorable phenotypic trait is a result of altered expression of said gene in response to the presence of the outer stimulus. Said altered gene expression is brought about by a sensor system in response to the presence of the outer stimulus.

The term “sensor system” used throughout the present specification and the appended claims shall mean a system comprising one or more components, which in one or more steps bring about altered expression of said gene in the presence of an outer stimulus. Such a system may comprise a number of sensory and regulatory entities such as for example promoters, regulatory elements, enhancers, regulatory proteins, antisense-RNA, transport- and receptor proteins and other parts of a signal transduction machinery as well as physico-chemical conditions such as pH etc. A sensor system may comprise one or any combination of such entities.

In a preferred embodiment of the invention, the sensor system comprises a regulatory element. In a further preferred embodiment of the invention the regulatory element comprises a metal response element (MRE) with the sequence TGCACCC, TGCACGC, TGCACAC or TGCGCAC (Scudiero et al. 2001).

In another preferred embodiment of the invention, the sensor system comprises a promoter, the activity of said promoter being affected by the presence of the outer stimulus. In a further preferred embodiment of the present invention said promoter is operatively coupled to the gene. In a most preferred embodiment of the present invention, the promoter is chosen from the group of Arabidopsis thaliana gamma-glutamylcysteine synthetase (X80377, X81973 and X84097), Arabidopsis thaliana phytochelatin synthase (PCS1, AF093753), Arabidopsis thaliana IRT1, and IRT2 metal transporters (U27590 and T04324), Arabidopsis thaliana AtPCS1, and AtPCS2 (W43439, and AC003027), Soya bean feritin (M64337, and M58336).

It will be obvious to a person skilled in the art that it is possible to develop a reporter system for plants according to the present invention, in which the phenotypic trait is the consequence of altered expression of more than one gene without deviating from the gist of the invention. Consequently such reporter systems are within the scope of the present invention.

In another preferred embodiment of the invention, the gene or genes is involved in the production of a visible colour change in plants. In a more preferred embodiment of the invention, the gene or genes is involved in phenylpropanoid metabolism, the biosynthesis of pigment, the biosynthesis of flavonoids or the biosynthesis of anthocyanins. In a most preferred embodiment of the invention the gene is chalcone synthase (CHS), chalcone isomerase (CHI) or dihydroflavonol reductase (DFR).

The term “involved in” as used in the paragraph above and the appended claims 9-13, shall comprise both the structural genes of the relevant metabolic pathway as well as genes involved in the regulation of said pathway.

Throughout the specification and the appended claims a number of specific genes, such as e.g. CHS, corresponding mutants such as e.g. tt4 and transcription factors such as PAP1 and PAP2 are referred to. This terminology is used in Arabidopsis thaliana. Equivalent genes which encode proteins with similar or identical biological function, corresponding mutants and transcription factors can be found in other plant species under different names. It is obvious that a person skilled in the art is able to develop reporter systems based on these components without deviating from the gist and the scope of protection of the present invention.

In a further aspect of the present invention concerning the reporter system for plants, functional copies of the endogenous gene or genes are rendered non-functional. Depending on the nature of the actual reporter system according to the present invention it may be necessary or advantageous to eliminate or reduce the activity of endogenous gene products which may interfere with the development of a distinct phenotype. If for example the actual reporter system is based on a chimeric gene comprising a coding sequence of a non-essential plant gene and a promoter of different origin, the endogenous plant gene may be rendered non-functional in order to obtain a more distinct phenotype in the presence of an analyte. Genes can be rendered non-functional by a number of methods known to a person skilled in the art (Sambrook et al. 1989) and such genes may be introduced in plants by transformation or crossing.

Accordingly, in a preferred embodiment of the present invention, the reporter system for plants furthermore comprises mutation of genes involved in the production of pigment. In a more preferred embodiment of the present invention, the reporter system for plants furthermore comprises mutation of genes involved in the flavonoid biosynthesis pathway, involved in the formation of tetrahydroxychalcon/chalcone synthesis or involved in the formation of 2S-flavanones, narringenein and ligquritigenin. In a most preferred embodiment of the present invention, the reporter system for plants furthermore comprises mutation of the CHI gene (tt5 mutant) or the CHS gene (tt4 mutant).

In a further aspect of the present invention concerning the reporter system for plants, the expression of transcription factors is furthermore altered. Transcription factors are proteins involved in transcriptional regulation. By altering the expression of these it may be possible to optimise the reporter system according to the present invention in order to obtain a more distinct phenotypic trait. If for example transcription factors positively regulating a pathway are overexpressed, and a reporter system based on a gene encoding one of the enzymes from said pathway is present in a null mutant, the expression of the reporter gene in the presence of an outer stimuli, may give rise to more end-product due to the overexpression of said transcription factors and consequently a more distinct phenotype. An example is the trancription of genes involved in flavonoid biosynthesis which are under positive regulation and directed towards the production of anthocyanins; the system is developed in a null background tt4 and/or tt5 mutant in which no anthocyanins are produced since their biosynthesis are blocked.

By complementation of the mutants i.e. inserting the CHS and/or the CHI gene under the control of a specifically regulated promotor and/or regulatory element(s), the production of anthocyanins will be controlled and a visible phenotype appears as a result of the specific stimulus which induce said promoter.

In a preferred embodiment of the present invention, the reporter system for plants furthermore comprises an altered expression of transcription factors containing a Myb domain. In a more preferred embodiment of the present invention, the reporter system for plants furthermore comprises an altered expression of transcription factors PAP1 and/or PAP2. In a further preferred embodiment of the present invention, the reporter system for plants also comprises overexpression of transcription factors. In a most preferred embodiment of the present invention, the reporter system for plants furthermore comprises overexpression of transcription factors PAP1 and/or PAP2.

When altering the expression of transcription factors, the choice of promoter may vary. Often a strong and constitutively expressed promoter, such as for example the 35S promoter or the dual promotor (Velten & Schell 1985 ) will be chosen if the transcription factor is to be overexpressed, but an inducible promoter which is responsive to the outer stimulus may prove advantageous if constitutive expression proves to be disadvantageous

Accordingly, in a preferred embodiment of the present invention, the reporter system for plants comprises overexpression of transcription factors which is controlled by an inducible promoter.

In another preferred embodiment of the present invention, the reporter system for plants comprises overexpression of transcription factors which is controlled by a constitutive promoter.

In a more preferred embodiment of the present invention, the reporter system for plants comprises overexpression of transcription factors which is controlled by the 35S promoter. In a further preferred embodiment of the present invention, the reporter system for plants comprises overexpression of transcription factors which is controlled by a dual promoter.

The outer stimulus may in principle be present either in the air, water or soil coming into contact with a plant carrying a reporter system of the present invention. The purpose of applying a reporter system of the present invention may be to identify the location and possibly the concentration and identity of either harmful substances, such as e.g. pollutants, or substances with may be beneficial, such as e.g. valuable metals.

Accordingly, in a preferred embodiment of the present invention, the reporter system for plants comprises one or more genes with an altered expression in the presence of inorganic pollutants. In a more preferred embodiment of the present invention, the reporter system for plants comprises one or more genes with an altered expression in the presence of heavy metals. In a most preferred embodiment of the present invention, the reporter system for plants comprises one or more genes with an altered expression in the presence of a heavy metal belonging to the group of Cu, Zn, Cd, Hg, Pb, Co, Cr, Ni, As, Be, Se, Au, Ag.

In another preferred embodiment of the present invention, the reporter system for plants comprises one or more genes with an altered expression in the presence of organic pollutants. In a more preferred embodiment of the present invention, the reporter system for plants comprises one or more genes with an altered expression in the presence of nitrogen-containing organic compounds. In a further preferred embodiment of the present invention, the nitrogen-containing compound contains NO2, NO3, NH2 or NH3. In a further preferred embodiment of the present invention, the reporter system for plants comprises one or more genes with an altered expression in the presence of a nitrogen-containing compound that was part of an explosive. In a most preferred embodiment of the present invention, the reporter system for plants comprises one or more genes with an altered expression in the presence of a nitrogen-containing compound that was part of an explosive.

The terms “pollution”, “soil pollution” or “polluted soil” as used throughout this specification and the appended claims shall be taken to mean any content of inorganic or organic compounds in the soil which is higher than what must be considered normal for that geographic area. It is not limited to compounds which may be considered harmful, but includes also compounds which may be useful or valuable if they are comprised by the above definition.

When the expression of a gene is altered due to the presence of a compound such as e.g. a pollutant, the interaction may be direct or indirect. By direct interaction the pollutant exerts the effect in the form in which it is found in the soil directly on the expression of the gene. By indirect interaction the pollutant is converted into a secondary stimulus that exerts an effect on the expression of the gene. The secondary stimulus may be a breakdown product of the pollutant, an entity in which the pollutant or us breakdown product is part, one or more entities (i.e. molecules, complexes or structural features) in which the pollutant or its breakdown products are not part or changes in the environment of the gene of physical or chemical nature. Such a conversion from pollutant to secondary stimulus may or may not involve an amplification step. The conversion from the primary stimulus to a secondary stimulus may require gene products encoded by genes not normally found in plants. When such genes are introduced into plants in a functional form they may facilitate said conversion in plants. Many genes of microbial origin possesses the capability to convert compounds which higher organisms can not and these may for example be introduced into the plant in order to facilitate the detection of a range of substances.

Accordingly, in a preferred embodiment of the present invention, the reporter system for plants comprises one or more genes with an altered expression in the presence of pollutants, wherein the expression of said gene or genes is altered directly by the presence of the pollutant.

In another preferred embodiment of the present invention, the reporter system for plants comprises one or more genes with an altered expression in the presence of pollutants, wherein the expression of said genes or genes is altered indirectly by the presence of the pollutant in a further preferred embodiment of the present invention, the pollutant is converted to a secondary factor in one or more steps and said secondary factor alter expression of said gene(s). In a more preferred embodiment of the present invention the conversion of the pollutant to a secondary factor is facilitated by microbial catabolic enzymes. In a most preferred embodiment of the present invention, the microbial enzyme is “TNT reductase” enabling the reduction of the pollutant and the release of NO₂ groups.

In another preferred embodiment of the present invention, the conversion of the pollutant to a secondary factor involves a cascade facilitating an amplification of stimulus.

In a preferred embodiment of the invention, the phenotypic trait may be assessed without performing an assay. In a more preferred embodiment of the invention, the phenotypic trait may be assessed by visual inspection. In a most preferred embodiment of the invention, the phenotypic trait is a colour.

In a further aspect of the present invention, the reporter system for plants furthermore comprises a bio-remediation system. The bio-remediation system may comprise the breakdown of the pollutant by the plant and may involve genes of e.g. microbial origin which encodes products fascilitating the breakdown. The bio-remediation system may also comprise accumulation of the pollutant in the plant or part of the plant whereby its removal is facilitated by removing the plants. In this case the pollutant may also subsequently be extracted from the plants if e.g. it is of sufficient value.

Accordingly, in a preferred embodiment of the invention, the bio-remediation system comprises the breakdown of the pollutant.

In another preferred embodiment of the invention, the bio-remediation system comprises accumulation of the pollutant, and thus fascilitates its removal. In a more preferred embodiment of the present invention, the accumulation is accomplished by the expression of one or a combination of heavy metal binding proteins and/or metal transport proteins. In a most preferred embodiment of the present invention, the heavy metal binding proteins and/or metal transport proteins comprise a gene belonging to the group of:

S. pombe gene encoding phytochelatin-synthetase(gene bank accession Y08414), Athyrium yokoscense AyPCS1 mRNA for phytochelatin synthase (AB057412), Arabidopsis thaliana putative phytochelatin synthase (AY039951), Arabidopsis thaliana phytochelatin synthase (CAD1, AF135155), Arabidopsis thaliana putative metallothionin-I gene trancription activator (AY04594), Arabidopsis thaliana phytochelatin synthase (PCS1, AF093753), Arabidopsis thaliana IRT1 and IRT2 metal transporters (U27590 and T04324), Arabidopsis thaliana AtNramp1, 2, 3 and 4 metal transporter (AF165125, AF141204, AF202539, and AF202540), Brassica juncea mRNA for phytochelatin synthase (pcs1 gene AJ278627), Euphorbia esula cDNA similar to phytochelatin synthetase-like protein (BG459096), Lycopersicon esculentum (Tomato crown gal) I similar to Arabidopsis thaliana putative phytochelatin synthetase (BG130981), Typha latifolia phytochelatin synthase (AF308658), Zea mays phytochelatin synthetase-like protein (CISEZmG, AF160475), Thalaspi caerulescens ZNT1 heavy metal transporter (AF 133267)

The heavy metal binding proteins and/or metal transport proteins may be expressed from both constitutive promoters, such as e.g. the 35S promoter, or an inducible promoter which responds to the presence of the pollutant as long as a sufficient amount of the proteins are expressed to obtain the desired capacity to accumulate the pollutant.

In a further aspect of the present invention, a genetically modified plant carrying a reporter system according to the present invention is provided.

The term “genetically modified plant” as used throughout this specification and the appended claims shall be taken to mean a plant which has a genetic background which is at least partially due to the use of genetic engineering. The progeny from such a plant or from crosses involving such a plant in the form of plants, seeds, tissue cultures and isolated tissue and cells, which carry at least part of the modification originally introduced by genetic engineering, are comprised by this definition.

In a preferred embodiment of the invention, the genetically modified plant is a monocotyledoneous plant.

In another preferred embodiment of the invention, the genetically modified plant is a dicotyledoneous plant.

In another preferred embodiment of the invention, the genetically modified plant is an annual plant.

In another preferred embodiment of the invention, the genetically modified plant is a biennial plant.

In another preferred embodiment of the invention, the genetically modified plant is a perennial plant.

In a more preferred embodiment of the invention, the genetically modified plant belongs to the Brassicaceae. In a further preferred embodiment of the invention the genetically modified plant belongs to the genus Arabidopsis.

In a most preferred embodiment of the invention, the genetically modified plant belongs to the group consisting of the following species: Brassica napus, B. rapa, and B. junceas, Brassica oleracea, Brassica napus, Brassica rapa, Raphanus sativus, Brassica juncea), Sinapis alba, Armoracia rusticana, Alliana petiolata, Arabidopsis thaliana, A. griffthiana, A. lasiocarpa, A. petrea, Barbarea vulgads, Berteroa incana, Brassica juncea, Brassica nigra, Brassica rapa, Bunias orientalis, Camelina alyssum, Camelina microcarpa, Camelina sativa, Capsella bursa-pastoris, Cardaria draba, Cardaria pubescens, Conringia orientalis, Descurainia incana, Descurainia pinnata, Descurainia sophia, Diplotaxis muralis, Diplotaxis tenuifolia, Erucastrum gallicum, Erysimum asperum, Erysimum cheiranthoides, Erysimum hieracifolium, Erysimum inconspicuum, Hesperis matronalis, Lepidium campestre, Lepidium densiflorum, Lepidium perfoliatum, Lepidium virginicum, Nasturtium officinale, Neslia paniculata, Raphanus raphanistrum, Rorippa austriaca, Rorippa sylvestris, Sinapis alba, Sinapis arvensis, Sisymbrium altissimum, Sisymbrium loeselii, Sisymbrium officinale, Thiaspi arvense, and Turritis glabra.

In a further aspect of the present invention, a process for detection of an analyte is provided comprising the steps of:
Introduction of seeds from a genetically modified plant according to the present invention.
Monitoring the phenotype of the resulting plants and,
Optionally the plants degrade the analyte as a bioremediation step or, if they accumulate the analyte, may be removed as a bioremediation step.

Plant seeds can be introduced by means of conventional methods for seed spreading, either manually or by applying a machine. In a preferred embodiment of the present invention the seeds are suspended in a solidifying substance such as agar or “dry water” which is frequently used as a “controlled release tool” for water in agriculture in dry areas. This will secure the supply of water nutrition and aid in keeping the seeds in place and evenly distributed.

In a preferred embodiment of the present invention, the analyte detected by said process is a pollutant.

In a further preferred embodiment of the present invention, the pollutant detected by said process is an inorganic pollutant.

In a further preferred embodiment of the present invention, the pollutant detected by said process is a heavy metal.

In a most preferred embodiment of the present invention, the pollutant detected by said process is a heavy metal from the group Cu, Zn, Cd, Hg, Pb, Co, Cr, Ni, As, Be, Se, Au, Ag.

In another preferred embodiment of the present invention, the process is able to detect a concentration of heavy metal of at least from 0.00025, such as 0.0005, e.g. 0.001, such as 0.0015, e.g. 0.002, e.g. 0.0025, such as 0.003, e.g. 0.004, e.g. 0.005, such as 0.006, e.g. 0.007, such as 0.008, e.g. 0.009, such as 0.01, e.g. 0.02, such as 0.03, e.g. 0.04, such as 0.05, e.g. 0.06, such as 0.07, e.g. 0.08, such as 0.09, e.g. 0.1, such as 0.2, e.g. 0.3, such as 0.4, e.g. 0.5, such as 0.6, e.g. 0.7, such as 0.8, mM e.g. 0.9, such as 1, e.g. 2, such as 3, e.g. 4, such as 5, e.g. 6, such as 7, e.g. 8, such as 9 e.g. 10 mM.

In a further preferred embodiment of the present invention, the pollutant detected by said process is an organic pollutant.

In a further preferred embodiment of the present invention, the pollutant detected by said process is a nitrogen-containing compound.

In a most preferred embodiment of the present invention, the pollutant contains NO₂, NO₃, NH₂ or NH₃.

In another preferred embodiment of the present invention, the process is able to detect a concentration of a nitrogen-containing compound of at least from 0.00025, such as 0.0005, e.g. 0.001, such as 0.0015, e.g. 0.002, e.g. 0.0025, such as 0.003, e.g. 0.004, e.g. 0.005, such as 0.006, e.g. 0.007, such as 0.008, e.g. 0.009, such as 0.01, e.g. 0.02, such as 0.03, e.g. 0.04, such as 0.05, e.g. 0.06, such as 0.07, e.g. 0.08, such as 0.09, e.g. 0.1, such as 0.2, e.g. 0.3, such as 0.4, e.g. 0.5, such as 0.6, e.g. 0.7, such as 0.8, mM e.g. 0.9, such as 1, e.g. 2, such as 3, e.g. 4, such as 5, e.g. 6, such as 7, e.g. 8, such as 9 e.g. 10 mM.

In a further aspect of the present invention, the use of a genetically modified plant according to the present invention for the detection of an analyte and optionally for bioremediation is provided.

In a preferred embodiment, the genetically modified plant is used according to the present invention to detect a pollutant.

In a further preferred embodiment, the genetically modified plant is used according to the present invention to detect an inorganic pollutant.

In a further preferred embodiment, the genetically modified plant is used according to the present invention to detect the a heavy metal pollutant.

In a most preferred embodiment, the genetically modified plant is used according to the present invention to detect a heavy metal belonging to the group of Cu, Zn, Cd, Hg, Pb, Co, Cr, Ni, As, Be, Se, Au, Ag.

In another preferred embodiment of the present invention the genetically modified plant is used for the detection of heavy metal at a concentration of at least 0.00025, such as 0.0005, e.g. 0.001, such as 0.0015, e.g. 0.002, e.g. 0.0025, such as 0.003, e.g. 0.004, e.g. 0.005, such as 0.006, e.g. 0.007, such as 0.008, e.g. 0.009, such as 0.01, e.g. 0.02, such as 0.03, e.g. 0.04, such as 0.05, e.g. 0.06, such as 0.07, e.g. 0.08, such as 0.09, e.g. 0.1, such as 0.2, e.g. 0.3, such as 0.4, e.g. 0.5, such as 0.6, e.g. 0.7, such as 0.8, e.g. 0.9, such as 1, e.g. 2, such as 3, e.g. 4, such as 5, e.g. 6, such as 7, e.g. 8, such as 9 e.g. 10 mM.

In a further preferred embodiment, the genetically modified plant is used according to the present invention to detect an organic pollutant.

In a further preferred embodiment, the genetically modified plant is used according to the present invention to detect a nitrogen-containing compound.

In a most preferred embodiment, the genetically modified plant is used according to the present invention to detect a pollutant containing NO₂, NO₃, NH₂, NH₃.

In another preferred embodiment of the present invention, the genetically modified plant is used to detect a concentration of a nitrogen-containing compound of at least from 0.00025, such as 0.0005, e.g. 0.001, such as 0.0015, e.g. 0.002, e.g. 0.0025, such as 0.003, e.g. 0.004, e.g. 0.005, such as 0.006, e.g. 0.007, such as 0.008, e.g. 0.009, such as 0.01, e.g. 0.02, such as 0.03, e.g. 0.04, such as 0.05, e.g. 0.06, such as 0.07, e.g. 0.08, such as 0.09, e.g. 0.1, such as 0.2, e.g. 0.3, such as 0.4, e.g. 0.5, such as 0.6, e.g. 0.7, such as 0.8, mM e.g. 0.9, such as 1, e.g. 2, such as 3, e.g. 4, such as 5, e.g. 6, such as 7, e.g. 8, such as 9 e.g. 10 mM.

It is an aim of the present invention to provide plants which will facilitate the bioremediation of polluted soils to a degree which results in the soil having a content of pollutants which is less than the limitations set by the environmental standards of the law. By planting seeds from plants according to the present invention and removing the resulting plants this may be achieved. The plants may be grown at—and removed from—a particular location one or several times in order to reduce the content of the pollutant to the required maximum level. Accordingly in a preferred embodiment of the present invention plants are grown at a polluted site and subsequently removed, as many times as is necessary to obtain the desired reduction in the concentration of pollutants in the soil.

In a preferred embodiment of the present invention the use of the plants is able to remove at least 10%, such as 20%, e.g. 30%, such as 40%, e.g. 50%, such as 60%, e.g. 70%, such as 80%, such as 90%, e.g. 95%, such as 99% of a pollutant per plant generation.

In another embodiment of the present invention the harvested plant biomass can be processed in order to obtain useful or valuable compounds such as e.g. heavy metals.

A preferred embodiment of the invention is detection of heavy metal contaminated soil. This may involve that the area of interest has to be cleared of vegetation already present. This can be achieved by mechanical means such as cutters, or in combination with herbicides such as Roundup (Glyphosate). Once the soil has been cleared of vegetation the seeds have to be spared. This can be accomplished by e.g. using a seed dispenser or spread suspended in a solution of a gelling agent in order to secure the position of the seeds until they have germinated and are rooted in the ground. The area is maintained with water and nutrients if needed depending on the quality of the soil. A visual inspection may be conducted for example 5 weeks after germination of the seeds and areas in which the plants display a red colour marked. Samples of the soil from these locations can be analysed by conventional methods to establish the degree of contamination.

In another preferred embodiment of the invention the plants display a colour change when the polution is just above the limit at which re-mediation have to be performed. This allows the colouration of the plants to be used directly as an indication for the need for re-mediation of the soil prior to using this for other human activities.

In a most preferred embodiment of the invention the colour change observed in the plants is accompanied by an uptake of the contaminant based on the presence of metal binding proteins and or metal transporters. At the time of maximum concentrations of heavy metals in the parts of the plants which are above ground, the plant biomass is harvested and the collected for further processing. In one preferred processing the plant material is colleted and deposited on a secure landfill. In a more preferred embodiment the plant material is incinerated and the contaminate colleted from the smoke. This way the volume of material which have to be deposited on the landfill can be reduced. In a most preferred embodiment of the invention the plant material is fermented in a bioreactor and the sloughs treated by electrolysis in order to regain useful metals.

In another embodiment of the invention the seeds are spread on an area which potentially contains valuable metals. Areas with red plants indicate potential metal mining sites and the colour change in the plants which are used for this purpose should ideally change colour when the concentration of the metal is sufficient to allow a profitable extraction.

In another embodiment the plants are spread in closed squares and watered with wastewater. If the waste water contains heavy metals the plants change colour and steps to reduce the heavy metal concentration in the water are initiated. In a most preferred embodiment the waste water is filtered by passage through the area with plants. The plants used for this task should change colour just below the max uptake by those same plants and thereby indicating that they have reached the saturation limit and additional influx of contaminated water will no longer be re-mediated by the plants.

In an embodiment of the present invention the presence of explosives in a municipal is detected. Existing vegetation in the area which is to be monitored and cleared for explosives have to be removed. Conventional methods employ mechanical viecals for forming squares of 25 m×25 m. The perimeters are laid down by flails (i.e heavily armoured vehicles) and afterwards all vegetation is removed by cutters mounted on long arms of about 12.5 meters. When hitting a land mine the arm and cutter will typically be damaged and may be replaced. In addition herbicides may be used to clear an area of vegetation. In this embodiment of the invention, the seeds may be spread in a suspension of herbicide, colour and a gelling agent. The herbicide is used to keep unwanted vegetation down. A colour different from both red and green may be added in order too ease a control of seed spreading to all open areas by visual inspection. The gelling agent may be included to secure that the seeds remain at the position at which they were distributed, ensuring full coverage of the soil. After e.g. 5 weeks, the 25×25 meter squares are inspected and if red plants are identified in a square this particular square have to be cleared by conventional methods of demining. This embodiment is normally referred to as AR (area reduction). In another embodiment of the invention the plants are used for AQI (area quality insurances), where areas already cleared by conventional methods are re-screened to make sure that no mines were missed the first time. In another embodiment of the invention soil contaminated with explosives, such as ammunition factory's/deposits or mineral mining pits, is monitored. In this embodiment the area potentially contaminated is cleared for vegetation and seeds are spread. After e.g. 5 weeks the site is inspected for red plants. Soil below the red plants can be removed and treated in order to remove the contamination.

The invention is described in further detail in the following paragraphs. The applied materials and methods and the examples are included for illustrative purposes and are not to limit the scope of the present invention. It will be obvious to a person skilled in the art that other experimental procedures may be developed or applied without deviating from the gist or the scope of the present invention, and these will as a consequence be comprised by the present invention.

### EXAMPLES

### Methods & Materials

The basic techniques used in the molecular work generating the constructs was as described by Sambrook et al. 1989, and by the following protocols;

PCRs (Long-Range)

All long-range PCRs were set-up according to the scheme below as 100 μl reactions with reagents from PERKIN ELMER (GeneAmp XL PCR kit # No. 808-0192), nucleotides from Pharmacia Biotec dATP, dTTP, dGTP and dCTP; all at stock concentrations of 100 mM were diluted in milliQ H₂O prior to use.

Reactions were run on an Eppendorf mastercycler 5330.

| long-range | | | | | | | | | | | | | | | | |
|---|---|---|---|---|---|---|---|---|---|---|---|---|---|---|---|---|
| no. reactions | | 1 X | | 6 X | | 8 X | | 10 X | | 12 X | | 18 X | | 24 X | | Final konc |
| Lower mix | | | | | | | | | | | | | | | | |
| H2O | | 13 | ul | 81.25 | ul | 107.25 | ul | 133.25 | ul | 162.5 | ul | 240.5 | ul | 321.75 | ul | |
| 3.3X XL buffer | | 12 | ul | 75 | ul | 99 | ul | 123 | ul | 150 | ul | 222 | ul | 297 | ul | 1 X buffer |
| dATP 10 mM | | 2 | ul | 12.5 | ul | 16.5 | ul | 20.5 | ul | 25 | ul | 37 | ul | 49.5 | ul | 200 uM |
| dTTP 10 mM | | 2 | ul | 12.5 | ul | 16.5 | ul | 20.5 | ul | 25 | ul | 37 | ul | 49.5 | ul | 200 uM |
| dGTP 10 mM | | 2 | ul | 12.5 | ul | 16.5 | ul | 20.5 | ul | 25 | ul | 37 | ul | 49.5 | ul | 200 uM |
| dCTP 10 mM | | 2 | ul | 12.5 | ul | 16.5 | ul | 20.5 | ul | 25 | ul | 37 | ul | 49.5 | ul | 200 uM |
| Primer 1 1 uM | | | | | | | | | | | | | | | | |
| Primer 2 1 uM | | | | | | | | | | | | | | | | |
| Mg(OAC)2 25 mM | | 4.4 | ul | 27.5 | ul | 36.3 | ul | 45.1 | ul | 55 | ul | 81.4 | ul | 108.9 | ul | 1.1 mM |
| | Volumer | 40 | ul | | | | | | | | | | | | | |
| Upper mix | | | | | | | | | | | | | | | | |
| H2O | | 39 | ul | 243.75 | ul | 321.75 | ul | 399.75 | ul | 487.5 | ul | 721 | ul | 965.25 | ul | |
| 3.3X XL buffer | | 18 | ul | 112.5 | ul | 148 | ul | 184.5 | ul | 225 | ul | 333 | ul | 445.5 | ul | |
| Template | | x | | x | | x | | x | | x | | x | | x | | |
| rTth polymerase | | 2 | ul | 12.5 | ul | 16.5 | ul | 20.5 | ul | 25 | ul | 37 | ul | 49.5 | ul | 4 units |
| | Volumer | 60 | ul | | | | | | | | | | | | | |

Melting of Wax Overlayer

1) 80° C. 5 min.

2) 25° C. 5 min.

3) End.

Standard Long-Range Program

1) 94° C. 1 min.

2) Loop 16

3) 94° C. 30 sec.

4) 68° C. 10 min.

5) Next 2

6) Loop 14

7) 94° C. 30 sec.

8) 68° C. 10 min.+(15 sec. extension)

9) Next 6

10) 72° C. 10 min.

11) 6° C. soak 30 sec.

12) End

PCR (Tag DNA Polymerase)

All Taq PCR reactions were set-up according to the scheme below in 100 μl reactions. Taq was from GibcoBRL life technologies # 18038-026, and nucleotides from Pharmacia Biotec, dATP, dTTP, dGTP and dCTP; all at stock concentrations 100 mM and have been diluted in milliQ H₂O for use. Reactions run on an Eppendorf mastercycler 5330.

| Taq-PCR | | | | | | | | | | | | | | | | |
|---|---|---|---|---|---|---|---|---|---|---|---|---|---|---|---|---|
| No. reactions | | 1 X | | 6 X | | 8 X | | 10 X | | 12 X | | 18 X | | 24 X | | Final konc |
| H2O | | 55.5 | ul | 346.8 | ul | 457.8 | ul | 568.8 | ul | 693.7 | ul | 1026.7 | ul | 1373.6 | ul | |
| 10X buffer | | 10 | ul | 62.5 | ul | 82.5 | ul | 102.5 | ul | 125 | ul | 183 | ul | 247.5 | ul | 1 X buffer |
| dNTP Mix (1.25 mM) | | 16 | ul | 100 | ul | 132 | ul | 164 | ul | 200 | ul | 296 | ul | 396 | ul | 200 mM |
| Primer 1 300 ng | | | | | | | | | | | | | | | | 300 ng |
| Primer 2 300 ng | | | | | | | | | | | | | | | | 300 ng |
| MgCl (25 mM) | | 6 | ul | 37.5 | ul | 49.5 | ul | 61.5 | ul | 75 | ul | 111 | ul | 148 | ul | 1.5 mM |
| template | | | | | | | | | | | | | | | | |
| Taq | | 0.5 | ul | 3.12 | ul | 4.12 | ul | 5.12 | ul | 6.25 | ul | 9.25 | ul | 12.37 | ul | 2.5 units |
| | volume | 100 | ul | | | | | | | | | | | | | |

Tag Standard Program for PCR on Plasmid DNA: 60° C.

1) 95° C. 3 min.

2) Hold waiting for key. (Add Taq)

3) 30 loops.

4) 94° C. 1 min.

5) 60° C. 2 min. (Can be adjusted 50-60° C. depending on primers and template)

6) 72° C. 1 min.

7) Next step 4.

8) 6° C. 30 sec.

Bacterial Work

E. coli Competent Cells (Hannahan Method)

1) Streak bacteria on fresh plates and grow o/n.

2) Pick 5-6 fresh colonies and dispense in Eppendorfs containing 1 ml SOB.

3) Use 1 ml to inoculate 100 ml SOB in a 1 l. flask. Grow at 37° C. for 2-3 h to OD595=0.2 (low density is critical).

4) Collect cells in four 50 ml disposable tubes at 2500 rpm for 15 min. at 4° C. Decant the supnatant and invert tubes to drain excess liquid. Resuspend pellet in 8 ml RF1/tube (⅓ vol.).

5) Place cells on ice for 15 min.

6) Collect cells at 2500 rpm at 4° C.

7) Decant supnatant and invert to drain. Resuspend in 1 ml RF2/tube ( 1/25 vol.). Place on ice for 15 min.

8) Pre-chill 40 eppendorf tubes (−80° C.). Aliquot 40 μl cells to each tube and freeze immediately in liquid nitrogen. Store at −80° C.

SOB Medium 500 ml

10 g Bactotryptone

2.5 g Yeast Extract

292 mg NaCl

0.9 g KCl

After autoclaving, add 5 ml of filter sterilized (0.22 μm filter) 1 M MgCl2 and 5 ml of a 1 M MgSO₄ (also filter sterilized), both to final concentration of 10 mM.

RF1 100 ml

1.2 g RbCl

0.99 g MnCl—4H₂O

3 ml of a 1M KOAc, pH=7.5 (adjusted with NaOH)

0.15 g CaCl—2H₂O

15 g Glycerol

Adjust pH to 5.8 with filter sterilized (0.22 μm filter) 0.2 M OAc.

RF2 50 ml

60 mg RbCl

1 ml of 0.5M MOPS, pH=6.8 (adjusted with NaOH).

0.55 g CaCl—2H₂O

7.5 g Glycerol

Adjust pH to 6.8 with filter sterilized (0.22 μm filter) NaOH.

E. coli Transformation

1) Thaw competent cells (−70° C. stored) on ice, invert to mix.

2) Add 150 μl cells to DNA samples in 13 ml tubes on ice.

3) Incubate 25 min. on ice with occasional mixing.

4) Heat shock 5 min., 37° C.

5) Incubate on ice for 5 min.

6) Add 1 ml LB without antibiotics, shake 1 h 37° C.

7) Spin 30 min., aspirate to 200 μl, plate 100 μl, store the rest at 4° C.

For blue/white screen, spread IPTG and X-Gal on plates before starting transformation.

200 μl 100 mM IPTG (0.2 g to 8.3 ml H₂O, 0.22 μm filter sterilized).

62.5 μl 4% X-Gal (0.4 g to 10 ml DMF, 0.22 μm filter sterilized).

Store both at −20° C., best if aliquoted. Do not mix together before use.

Positive control uses 10 ng supercoiled plasmid.

Miniprep—Alkaline Lysis

1) 1.5 ml over night culture to eppendorfs, spin 1 min., aspirate supernatant

2) Resuspend by vortex 5 min. RT in 100 μl miniprep solution 1 MPS1

3) +200 μl MPS2, invert tubes rapidly 3 times, inc 5 min. on ice

4) +150 μl MPS3, vortex upside down 10 min., inc 5 min. on ice

5) Spin 5 min. RT

6) Transfer to eppendorfs—7a) for sequencing

7) PCHCl3 ext

8) Spin 2 min. RT

9) Transfer eppi

10) +900 μl EtOH

11) Inc 2 min. RT

12) Spin 5 min. RT

13) Aspirate

14) 70% EtOH wash & spin

15) Aspirate, speedvac

16) Resuspend in 50 μl TE, use 2 for digests
7a) +900 μl EtOH
8a) Spin 5 min. RT
9a) Aspirate
10a) +1 ml 70% EtOH, spin
11a) Aspirate, resuspend in 200 ml TE, 2 mg RNAseA, incubate for 15 min. at 37° C.
12a) Phenol/CHCl3 extract, add 20 ml 3 M NaOAc, EtOH ppt, 70% wash
13a) Resuspend in 30 ml TE
14a) See Sequenase protocol for denaturation
16) Resuspend in 50 μl TE

Solutions:

| | stock | | |
|---|---|---|---|
| MPS1, frozen | | 50 | ml |
| 50 mM glucose | 2 M | 1.25 | ml |
| 10 mM EDTA | 0.25 M | 2 | ml |
| 25 mN Tris 8 | 1 M | 1.25 | ml |
| MPS2, fresh | | 10 | ml |
| 0.2 N NaOH | 10 N | 200 | μl |
| H₂0 | — | 8.8 | ml |
| 1% SDS | 10% | 1 | ml |
| MPS3 | | 100 | ml |
| 60 mM KOAc | 5 M | 60 | ml |
| 1.2 M HAc | | 11.5 | ml |
| H₂O | — | 28.5 | ml |

LB Liquid for Solid Add 14 g/l Difco Bacto Agar

1) 22 g/l Lainer broth GibcoBRL

2) Add up to 1 l. milliQ H₂O

3) Autoclave (120° C. 20 min.)

4) Add antibiotic just prior to use (media at room temperature)

5) (For LB plates add 15 g/l Difco bacto agar)

All Constructs were transferred to Agrobacterium by electroporation

Agrobacterium Competent Cells

1). Inoculate 2 ml YEP+antibiotics, with toothpick and grow at 28° C. over night on a shaker. ABI—50 KAN & 25 Chlor, gv3101—25GEN

2). Transfer the o/n culture to 200 ml YEP in a sterile 500 ml flask and shake at 250 rpm until the OD is 0.3 (4-5 h)

3). Spin in sterile 50 ml screw cap tubes 4° C. 5 krpm 10 min. Check to make sure cells are pelleted, if not repeat at higher speed.

4). Aspirate supernatant, resuspend pellet in 20 ml ice cold 1 mM HEPES pH 7 (sterile filtered), respin.

5). Repeat 4. two more times.

6). After aspirating, resuspend pellet in 2 ml ice cold 10% glycerol (sterile filtered).

7). Immediately dispense in 40 μl aliquots in pre-chilled, sterile eppis, freeze in I N2 and store at −70° C.

Agrobacterium Electroporation

DNA Preparations

DNA for electroporation must be free of salt, RNA or protein. DNA (in TE buffer) should be first treated with RNase, then twice extracted with phenol/chloroform. This will remove protein and RNA. To remove salt, EtOH precipitate the DNA and wash twice with 70% ethanol. Resuspend the DNA at 0.4-1 μg/ml.

Electroporating

Electrocompetent bacterial cells, YEP media and DNA solutions must be kept on ice before mixing. Note that the following steps should be carried out in under 1 min. and that you should be wearing glasses and gloves.

16. mix 1-2 ml DNA (600 ng) with 40 ml cells.

17. Transfer the DNA/cell mixture to a cuvette on ice avoiding air bubbles by gently shaking the cuvette.

18. Dry outside of the cuvette with tissue paper and insert the cuvette into the cuvette chamber with notch facing towards you.

19. Close cuvette chamber lid.

20. Set Arm/Disarm to ARM (arm light comes on).

21. Set Charge/Pulse to pulse and the pulse light will come on briefly.

22. When pulse light is off, set Arm/Disarm to DISARM (arm light comes on) and remove cuvette.

23. With DNA/Agrobacterium mix still in cuvette, add 500 ml cold YEP (no antibiotics) and mix solution by gently pippeting up and down.

24. Transfer the cells to an eppi and incubate at 28° C. for 2-4 h.

25. Leave the electroporator with the switch in the PULSE position

26. Plate 200 ml on YEP+antibiotics.

27. Incubate at 28° C. and colonies will appear in 2-3 days.

Re-Using Cuvettes

Fill a used cuvette with 0.1 M H₂SO₄ and let it stand for 15 min. Rinse 6 times with dH₂0, then 2 times with 96% EtOH. Store well-covered in 70% EtOH.

Agrobacterium Miniprep

Agrobacterium wich was used for plant transformation was checked for the presence of the Ti plasmid as plant transformation and the analysis of transgenic plants is time consuming. The preferred method was to make an agrobacterium miniprep and to use PCR to determine that the cells contain the correct construct. PCR was preferred here because the Ti plasmid is single copy and barely visible on a agarose gel.

1) Grow cells overnight in 5 ml LB or YEP with antibiotics. For pMONs in ABI—50 μg/ml KAN, 50 μg/ml Spec, 25 μg/ml Chlor. For pBI types in gv3101—50 μg/ml KAN, 25 μg/ml GEN.

2) Transfer 1 ml cells to two microfuge tubes.

3) Centrifuge 45 sec. and remove the supernatant with aspiration.

4) Add 1 ml cells more to both tubes and repeat step 3.

5) Vortex the pellet, add 100 μl MPS1 solution, vortex again and incubate the tubes at room temperature for 5 min.

6) Add 20 μl of a 20 mg/ml lysozyme solution, vortex-spin 1 sec. and incubate 15 min at 37° C.

7) Add 200 μl MPS2 solution (freshly made), mix gently by turning the rack 3-4 times and incubate 5 min. on ice.

8) Add 150 μl MPS3, vortex for at least 10 sec. and incubate 5 min. on ice.

9) Centrifuge for 5 min. and transfer the supernatant to new tubes.

10) Add 400 μl phenol/chloroform/isoamyl alcohol (25:24:1), vortex, centrifuge for 5 min and transfer the supernatant to new tubes.

11) Repeat step 10.

12) Repeat step 10 with chloroform alone.

13) Add 300 μl isopropanol and incubate on ice for 10 min.

14) Centrifuge for 5 min. and wash pellet with 70% EtOH.

15) Dry pellet and resuspend the two tubes in a total of 50 μl TE-buffer+RNase, use 2 μl for a PCR, freeze the rest.

| | Stock | | |
|---|---|---|---|
| MPS1 for 50 ml | | | |
| 50 mM glucose | 1 M | 2.5 | ml |
| 10 mM EDTA | 0.5 mM | 1 | ml |
| 25 mM Tris pH = 8.0 | 1 M | 1.25 | ml |
| MPS2 for 10 ml | | | |
| 0.2 N NaOH | 10 N | 200 | μl |
| 1% SDS | 10% | 1 | ml |
| H₂O | | 8.8 | ml |
| MPS3 for 100 ml | | | |
| 5 M potassium acetate | | 60 | ml |
| glacial acetic acid | | 11.5 | ml |
| H₂O | | 28.5 | ml |

Following the transfer of the constructs to Agrobacterium the constructs were transformed into plants using the protocol below;

All constructs were transformed into Agrobateria thumefasiens and transferred to plants by vacuum infiltration

Vacuum infiltration using a modified protocol based on (Bechtold & Pelletier 1998).

Plant Growth:

1. Take seeds with a brush and place them into 8 cm square pots filled with soil. Don't compress the soil too much and water the pots thoroughly with 2-3 pot-vol to remove excess nutrients. Place 12-16 seeds in each pot. Place the pots in the cold room for two days before transferring them to the growth chamber. Grow the plants for three weeks in short days (10 hr or less) to get large plants and a greater seed yield. Transfer the pots to long days to induce bolting. Grow plants to a stage at which bolts are around 10 cm tall.

2. Clip off emerging bolts close to rosette leaves to encourage growth of multiple secondary bolts. Infiltration will be done 7 to 9 days after clipping (plants will be 10-15 cm high and the biggest of the inflorescence will have made the first tiny silique. Do not water the plants the day before vacuum infiltration.

Vacuum Infiltration:

3. Start a 4 ml agrobacterium culture (YEP+antibiotics) inoculated from a −800C stock or from a plate. Grow cells O/N to 48 h depending on the strain. Add this culture to 250 ml of YEP+antibiotics (A 250ml culture will give enough cells for infiltration of 6 pots). Grow the culture between O/N and 2 days (depending on the strain) to OD600=1.2-1.8. The culture will have a mother of pearl appearance (not lumpy or black).

4. Spin down agros at 5000 rpm for 10 min in 250 ml centrifuge bottles, resuspend in infiltration media to an OD600=0.8 in a minimum volume of 300 ml.

5. Poor the agro suspension into a beaker of an appropriate size (400 ml is ok). Place the beaker into the vacuum jar. Degass the solution by drawing vacuum until bubbles form. Place a paper towel under the beaker to avoid that the beaker gets stuck in the bottom of the vacuum jar.

6. Sprinkle the plants with water 5 min prior to infiltration (optional) and then invert plants into the culture solution. Make sure that all the flowers are submerged and leave 2 cm between the rosettes leaves and the agro suspension. Don't let the culture contact the rosette or soil as this could kill the plants. Avoid that the solution boils over when you pull the vacuum. Make sure that the soil is only moist, so that the water from the pots does not enter into the culture suspension (therefore we recommend not to water the plants the day before infiltration). Draw vacuum for 15-20 min for WS and 30 min for Col-0 at a pressure close to 0.05 Bar (we are using an oil pump).

7. Before removing the plants from the vacuum jar place a plastic bag over the pot and beaker. Pull out and remove plants from the beaker, lay pots on their side (to avoid that excess infiltration media runs down into the soil). Fold over the top of the plastic bag and staple them twice. The other possibility is to place the pots laying on their side into a tray and cover the whole box with saran wrap. Put them in a growth chamber for one night. Next day move them to the green house. Put the plants in vertical position and open the bags. Next day get rid off the bags. In case you have the plants in trays: put also the plants in vertical position and use sticks and saran wrap to make a kind of a tend around the plants. Next day remove the plastic. In hot summers, we recommend to give plants a shower after we have placed them in vertical position (the purpose of this is to remove the sugars from the infiltration media which decrease fungal infection).

8. Grow plants for approx. four weeks, keeping bolts from each pot together but separated from neighbouring pots.

9. When the siliques begin to turn yellow, place the pot on its side with the plants inside a big envelope. Leave them for one week to dry out and cut off the plants. Let the seeds dry in the envelope and clean them 10 days later (keep all the seeds from one pot together). Store the seeds in the cold room for one week before plating them.

Kanamycin Selection Protocol

1. Sterilisation of Seeds:

Aliquot seeds in 15 ml falcon tubes (approx 700 seeds/tube, you can estimate the amount of seeds by first drawing a square plate of 9 cm×9 cm on a paper and spreading the seeds on it). Add 10 ml of hypoclorite solution. Shake tubes for 10 min. Remove the solution and add 10 ml of 70% ethanol. Wait 2 minutes. Discard EtOH and wash seeds 2-3 times with 10 ml of sterile water. Resuspend seeds with 8 ml 0.7% top agar (no warmer than 55° C.)

2. Spread seeds onto selection plates (MS+Kan). Dry plates in laminar flow hood until the top agar has solidified.

3. Vernalize plates for two nights in the cold room at 4° C. Transfer the plates to the growth chamber (21° C. with continuous light).

4. After approx. 7 days transformants should be clearly identifiable as dark green plants with healthy green secondary leaves and roots that extend into the selective medium. Root growth is the most clear maker to identify transformants at an early stages.

To make sure that the transformants are positive transfer them to a new MS+Kan plate and leave them there for a few days (if they turn yellow is because they are false positives). Transfer the seedlings to soil.

If you have contamination on your plates at this step, transfer the transformants as early as possible to soil.

5. Grow the plants and collect the seeds.

Infiltration Media

½× Murashige&Skoog salts (SIGMA #5524)

1× B5 vitamines (1 ml of 1000× stock) (SIGMA; #G-2519) Gamborg's vitamine powder, to prepare the 1000× stock dissolve 11.2 g in 100 ml water.

5% sucrose

adjust to pH 5.7 before autoclaving

after autoclaving add:
Benzylamino Purine (BAP), 10 &micro; I per liter of a 1 mg/ml stock in DMSO. By adding the hormone just before use, you can keep infiltration media as a stock for at least one week prior to infiltration.
we recommend to add 0.01% silwet to the infiltration media to increase transformation efficiency especially for Landsberg and colombia ecotypes. (silwet is from LEHLE SEEDS, cat no VIS-01 VAC-IN-STUFF (sliwet L-77))
   Kanamycin/Hygromycin Selection Protocol:

1. Sterilize seed.

2. Plate seed by resuspending in sterile, 7% 55 C top agar (125 seeds pr ml) and pour/swirl onto selection plates (rather like plating phage). Dry plates in laminar flow hood until seed no longer flows when plate is tipped. For normal 9×9 cm plates, 625 seeds is good (5 ml). Higher density could make it difficult to spot positive plants because antibiotic selection will be less effective.

3. Vernalize plates for two nights in cold room 4° C. Move plates to growth chamber.

4. After about 7 days, transformants should be clearly identifiable as dark green plants with healthy green secondary leaves and roots that extend over and into the selective medium. Root growth is the best marker.

5. Transplant plantlets to soil, grow and collect seed. Transplanting success is improved by a) using 7% agar in selection plates because it is easy to pull the roots out without agar lumps or breaking, b) saturating soil with water after transplanting, and c) growing plants under a dome (use Aracon seed collector to maintain high humidity for the first day or two. If you break the root, put plantlet onto a new selection plate for a few days before transplanting.

Selection Plates:

1× Murashige&Skoog salts

1% sucrose

adjust pH 5.7 with 1M KOH.

0.7% Difco agar.

autoclave, cool, and add:

1× MS vitamines (SIGMA #M-7150. take 1 ml of 1000× stock prepared by dissolving 10.3 gr in 100 ml of water.)

antibiotic (kanamycin 50 mg/B).

Top Agar

100× Murashige&Skoog salts.

1% sucrose.

adjust pH 5.7 with 1M KOH.

0.7% Difco agar.

autoclave.

before use: boil in the microwave and keep in water bath at 50-55° C.

YEP Media (Liquid):

10 g/l Bacto peptone (Difco)

10 g/l Yeast extract (Difco)

5 g/l NaCl

For YEP plates add 15 gr/l Difco bacto agar.

Hypoclorite Solution:

for 50 ml:

4 ml Na Hypoclorite 15%

255 l Tween-20

water to 50 ml

LUC Imaging

Luciferase Assays CCD Camera.

The protocol was as described by (Meier et al. 2000).

Luciferin Preparation:

D-luciferin-potassium (Hemica ALTA Ltd #0572)

Stock: 50 μM (Mw 318.4) 0.159 g dissolved in H₂0 and aliquoted into eppendorfs 1 ml in each (store −80° C.)

Working concentration: 5 μM

Preparation of 10 ml Working Solution

1 ml of stock

9 ml H₂O

5 μl 20%Triton X 100

Filter sterilize (20 μm filters)

Ones working solution is made store at 4° C. for up to 2 weeks.

The luciferin is applied to the plates by spraying. For a 9 cm plate, use 200 μl working solution. This should be done in a flowhood.

All generated GMO plants was maintained under the following conditions

Soil and Growth Conditions

Soil mixture:
100 l. K-soil (weillbøll, Sweeden)
6 l. Perlite
6 l. Vermiculite
300 g Osmocote (Scotts 3-4 month realse time NPK 15-15-11)

Pots: 9×9 cm plastic pots, square for vacuum infiltration

Pots 4.5×4.5 plastic pots for single plants

For growing and collecting seeds of single plants. An Aracon system (# AS-0007 Betha Tech) was used.

Growth Conditions:

Tissue Cultures: 21° C. Room

Temperature: 21° C.

Humidity: 60%

Long day: 20 h/4 h light/dark.

Green Houses:

Temperature:

Humidity: 95%

Long Day 13 h

Growth Chamber 1:

Short day 8 h

Temperature: 20° C.

Humidity: 60%

Growth Chamber 2:

Long day 14 h

Temperature: 20° C.

Humidity: 60%

Crossing Arabidopsis Plants

(Flower Emmansculation and Flower Preparation for Fertilization)

Prior to performing the above experiments, maturing flowers must be present in the bolting Arabidopsis plants.

1. Preparation of recipient flower (ovary).

The objective is to remove all the flower parts except the ovary.

Choose an inflorescence and remove all the flowers that are too young (too small) and the ones that already show white petals (opening flowers will tend to have started self-fertilization). Cut both too young and too old flowers from inflorescence, leaving 3-10 flowers in the middle to work with

Cut of all other plant parts in the immediate vicinity, specually siliques. The idea is to have as free a work environment as possible.

While cutting parts of from flowers, DO NOT tare parts off. Flowers are delicate and be easily damaged. Practice will give a good feel for how much they can take.

This procedure can be done using very fine forceps: INOX1.

In between flowers, clean forceps by dipping them in 95% ethanol followed by distilled water.

Use a kim-wipe as surface while viewing the flowers on a dissecting scope. This helps in holding the flower parts to the paper and not the forceps.

2. Obtain pollen.

Obtain fully mature flowers and remove the stamens. Use these stamens to brush the prepared ovaries. Repeat this at least twice to make sure there is plenty of pollen at the tip of the flower. This should be evident when looking at the ovaries through the dissecting scope as the pollen looks like a grainy brownish surface on top of the green ovary.

3. Label the cross accordingly and wrap the ovaries with Raynolds 905 sahran-wrap to make sure to cross contamination takes place.

4. Leave ovaries developing until they start yellowing before harvesting. If too dry, they may shed their seeds.

Selection Markers within the Plasmid Constructions

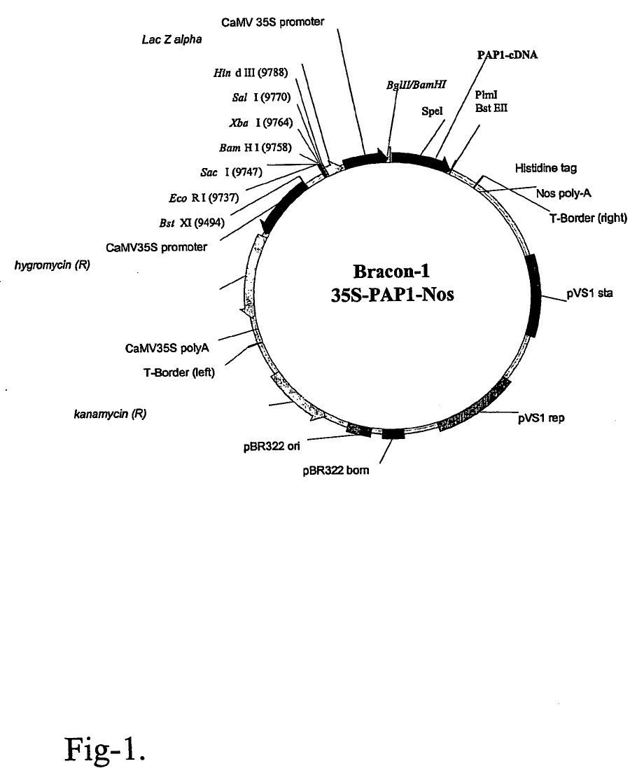

The antibiotic selection markers (kanamycin/hygromycin) were substituted with other selection systems (LUC, GFP) using homologous recombination (Court et al., 2002). The plasmids are illustrated with kanamycin/hygromycin as selection markers only (FIG. 1-FIG. 30).

### EXAMPLE 1

### Plasmid Constructions for the CHS-PAP Reporter System

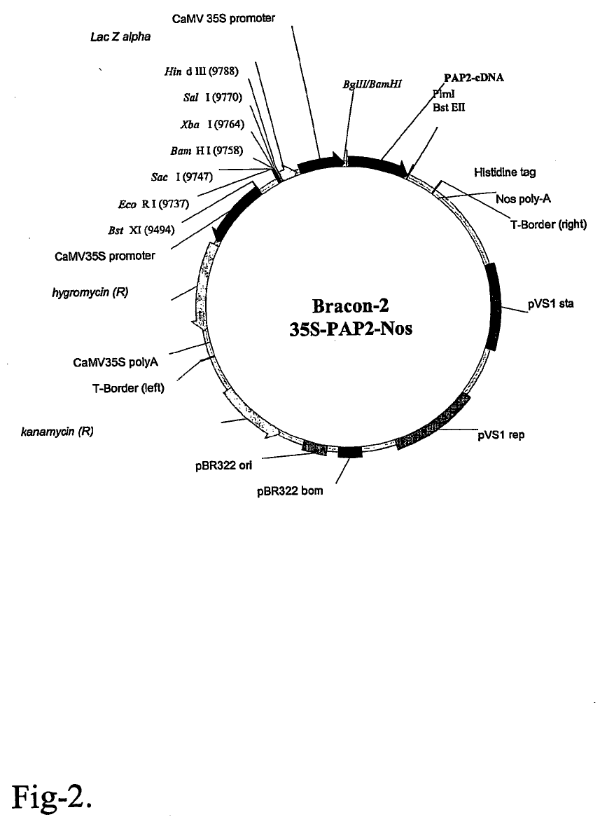

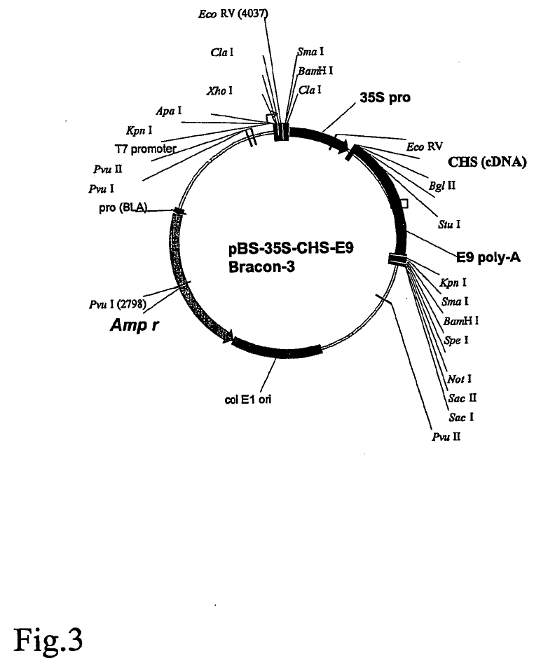

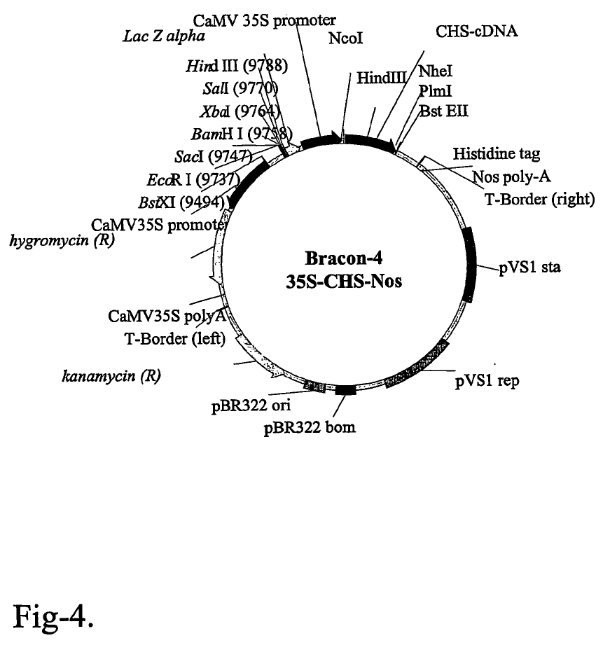

The pap1 (production of anthocyanin pigment 1, gene bank accession AF325123 ) and pap2 (production of anthocyanin pigment 2, gene bank accession AF325124) MYB transcription factors (Borevitz et al. 2000) cDNAs were obtained by LR-PCR (Long-range) using the RTth polymerase and the following primers pap1 FW 5′AAGGATCCATGGAGGGTTCGTCCAAAGGGCTGCGA 3′ and RW 5′AACCTAGGCTAATCAAATTTCACAGTCTCTCCATC 3′ and the PAP2 FW 5′AAGGATCCATGGAGGGTTCGTCCAAAGGGTGAGG 3′ and RW AACCTAGGCAGACTCCAAAGTTGCTCAACGTCAAACGC 3′ the amplified sequences was examined by restriction digestion and the obtained sequences were tailed and subsequently ligated into the pGEMT-easy vector (Promega kit #A1360). Positive clones were sequenced using an ABI Capillary Sequencer and the big dye system (#A016) in order to confirmed that the correct sequence was amplified and that no mistakes introduced. Both genes were excised using EcoRI and the resolving fragments blunted using Mung Bean nuclease these blunted, fragments were ligated to The Cambria transformations vectors 1302. The PAP1 (FIG. 1) and PAP2 (FIG. 2). was inserted 3 prime to the 35S promoter. The 1302 vector was previously prepared by digestion with BglII/NheI thereby excising the gfp*5 gene, the vector was blunted, and treated with CIP (Calf Intestinal Phospothase.) According to the manufacturers protocol. The CHS (naringenin-chalcone synthase) gene bank accession AY044331, encoding the ff4 protein. The CHS cDNA was obtained in a similar procedure as described for the PAP1 and PAP2 genes using FW primer 5′ATGGTGATGGCTGGTGCTTCTTCTT 3′ and RW 5′TTAGAGAGGAACGCTGTGCAAGAC 3′. The PCR product was tailed and ligated into the Pgem-Teasy vector. Subsequently the CHS gene was excised by digestion with Not I the purified fragment was blunted using Mung Bean nuclease and ligated into the Pbs35S-E9 cloning vector FIG. 3. This construct was generated for promoter cloning. Secondly a Cam 35S-CHS-E9 transformation construct was generated by excising the 35S-CHS-E9 cassette using Sma I and ligating the fragment into the cam1302 vector witch was cut Sma I and Cip'ed FIG. 4.

### EXAMPLE 2

### Plasmid Constructions for Heavy Metal Detection

GSH1 5′UTR

The following are given as an example for a heavy metal detection system but not limited to these heavy metal regulated promoters. The GSH (gamma-gutamylcystine-synthetase gene bank accession AF0682299) (Cobbett et al., 1994) 5′ UTR promoter) were obtained by LR PCR using the FW primer 5′GTGATATATAGCCATAATTGTGTT 3′ and RW 5′GTATATATAGCTCCTGCAATTATA 3′ The amplified sequence spacing 1185 bp from −1183 and to +2 the obtained fragment were tailed and ligated into the pGEM-T-easy vector and subsequently sequenced.

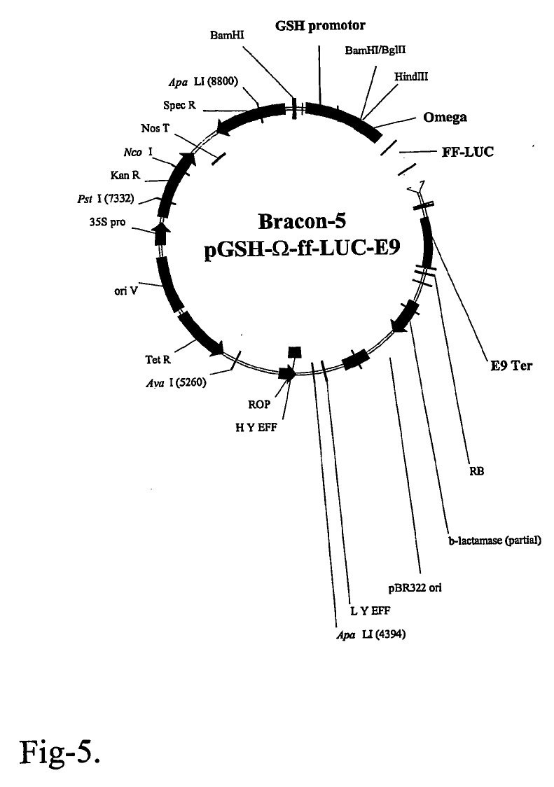

The GSH promoter fragment was inserted in front of the omega leader and the ff-LUC gene as a BamHI/BglII fragment in the BamHI cut and Cip (Vip11-Omeg-LUC vector). In order to examine the regulation of the promoter. FIG. 5.

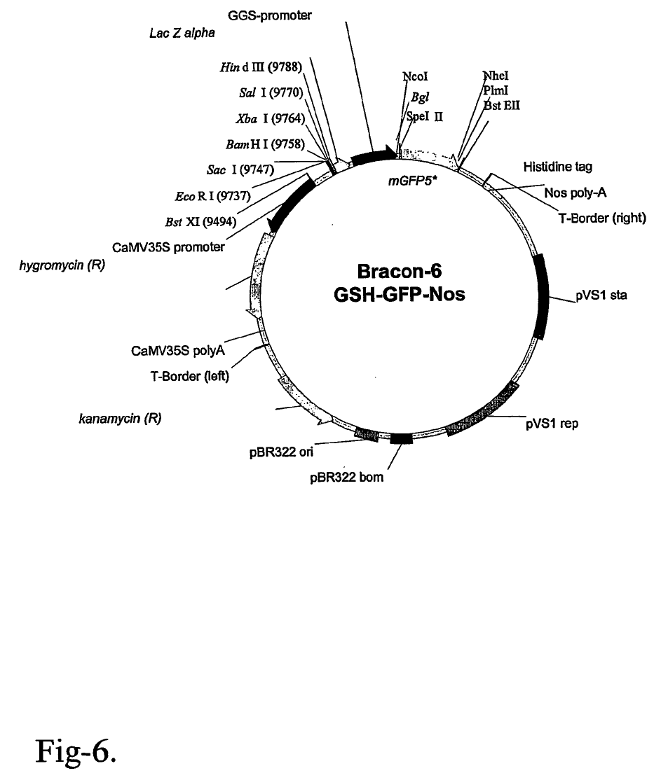

The GSH promoter fragment was excised as an Nco I/Sal I fragment from the Teasy vector. The cam 1302 vector was cut NcoI/SalI to release the 35S promoter leaving the GFP-Nos ready for ligation with the GSH fragment. Giving the construct GSH-GFP-Nos. FIG. 6.

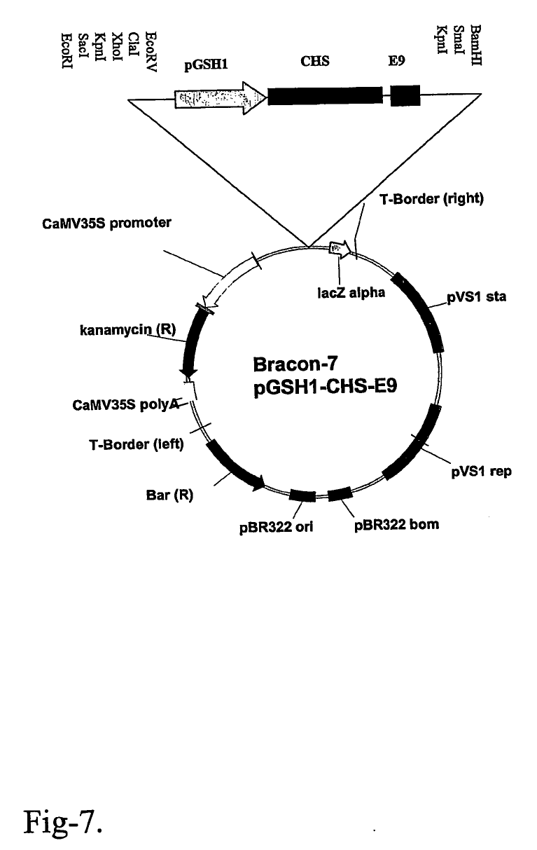

The GSH1promoter fragment was excised as an Nco I/Sal I fragment from the Teasy vector and blunted by Mung Bean nuclease. The blunt end fragment was inserted into the Stu I site giving the cassette pGSH1-CHS-E9. The cassette was released by digestion with KpnI and the fragment cloned into the Kpn I site in the cam2200 transformation vector FIG. 7.

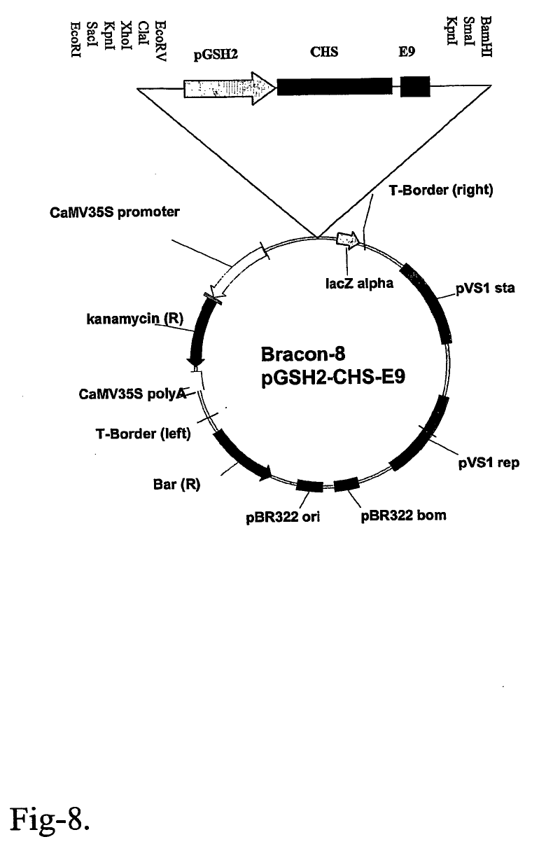

GSH5′UTR (Glutathione synthase, gene bank accession X83411) was amplified with the primer combination of FW 5′-GATATCAAGAGGATAAGAGGATTGTGTTGGA-3′ (EcoR V linker) and RW 5′-AGATCTCTTAAATGATCTCCCACACCTCAAA-3′ (Bgl II linker). The promoter fragment from −712 to −1 (711 bp) of pGSH2 was released from the pGEMT easy vector by digestion with EcoR Vl BglII. The obtained fragment was replacing the 35S promoter in the Bracon3 plasmid giving a Pbs-pGSH2-CHS-E9 cassette. The cassette was excised by digestion with Kpn this cassette was ligated into the Kpn I site in the cam2200 transformation vector. The following construct was generated in this way FIG. 8.

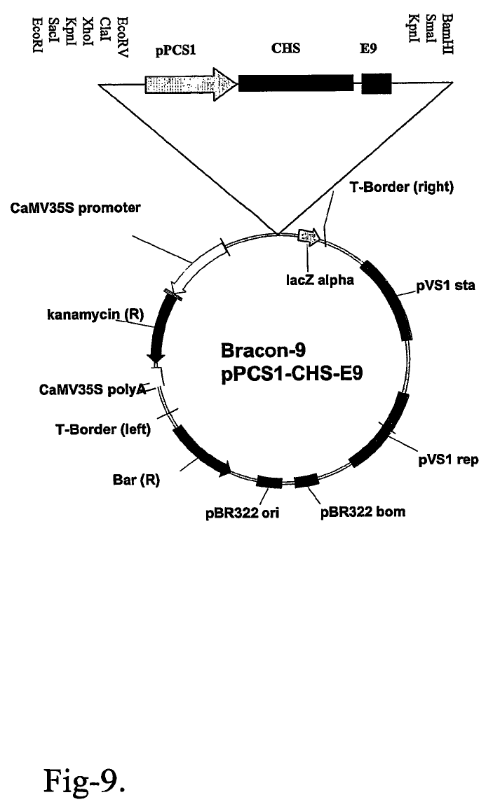

PCS1 5′UTR (gene bank accession AF461180) was amplified by LR-PCR from genomic DNA using the following primers FW 5′-GATATCAACTTTTTTGCTTCTCCTTTTTCAA-3′ (EcoR V linker) and RW 5′-AGATCTTTTTCACTGCTTGTTTTGGTATCTA-3′ (Bgl II linker) the obtained fragment from −915 to −1 (914 bp) was tailed and ligated into the pGem-Teasy vector and subsequently sequenced to confirm the correct gene was amplified. The insert was released by digestion using EcoR V and Bgl II. In forehand we had prepared the vector Bracon3 by existing the 35S promoter with EcoR V and Bgl II and gel purified the vector. The legation yielded a cassette Pbs pPCS1-CHS-E9 and this cassette was transferred to the cam2200 transformation vector by digesting the Pbs-pPCS1-CHS E9 plasmid with Kpn I and ligating the cassette into the Kpn I site of the Cam2200 vector. FIG. 9.

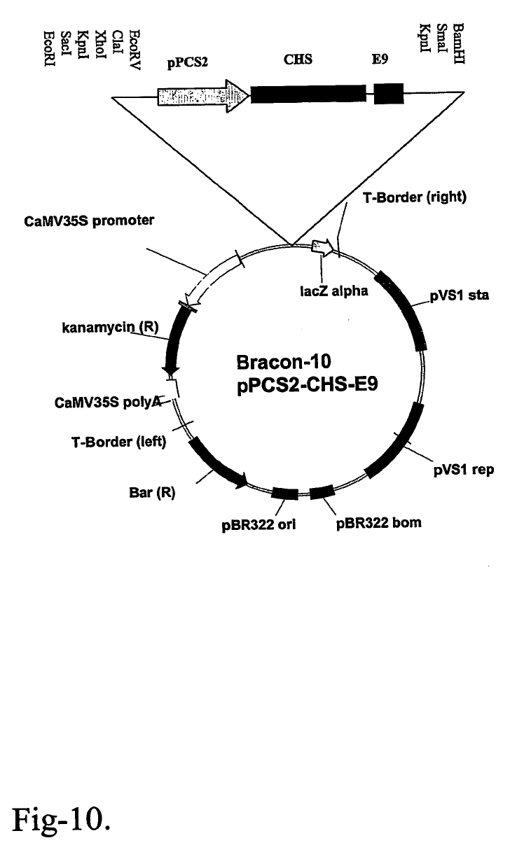

PCS5′UTR (gene bank accession AY044049) promoter was amplified from genomic DNA using a combination of the Fw primer 5′-GTTAACGATTCGACTCGGTCACGTGATATAC-3′ (Hpa I linker) and RW 5′-AGATCTGTCAGAGTTTGACTATGGAGCAAAC-3′ (Bgl II linker). The obtained fragment spading the genomic sequence from −875 to −2 (973 bp) was tailed and ligated into the pGEMT easy vector. The pPCS2 fragment was released by digestion with the restriction enzymes Hpa I and Bgl II. The Hpa I/Bgl II fragment was ligated into the Bracon3 plasmid thereby replacing the 35S promoter, witch was excised by cutting the Bracon3 plasmid with EcoR V and Bgl II and gel isolate the vector. The ligation gave the cassette Pbs pPCS2-CHS-E9 and this cassette was excised by digesting the plasmid by Kpn I and ligating the fragment into the Kpn I site of cam2200 TDNA vector. FIG. 10.

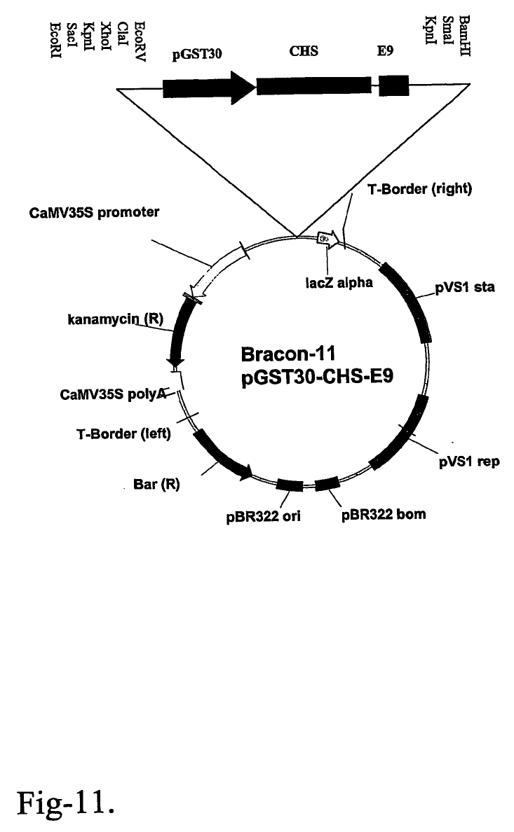

GST30 5′UTR (glutathione S-transferase family in Arabidopsis thaliana, homologue to the maize Bronze2 gene, gene bank accession AF288191) was amplified with the primer combination of FW 5′-GATATCATAATTATGTCAATCTTGCGTGTTT-3′ (EcoR V linker) and RW 5′-AGATCTTTTCTCTTCAAAATCCAAAACAGAG-3′ (Bgl II linker) The amplified product, from −1051 to −1 (1050 bp) was restriction checked and tailed and ligated into the pGEMT easy vector. In the next step the promoter fragment pGST30 was released by digestion with EcoR V and Bgl II this sticky end fragment was ligated into the EcoR V and Bgl II sits of Bracon3 already prepared by excising the 35S promoter with EcoR V and Bgl II and gel isolation the ligation gave the cassette pGST30-CHS-E9 and the cassette was moved into the transformation vector by Kpn I FIG. 11.

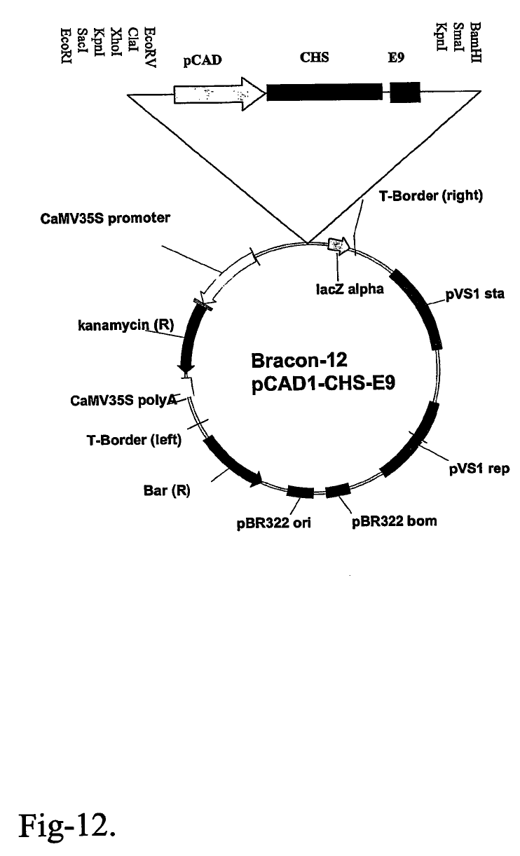

CAD1 5′UTR (Phytochelatin synthase, gene bank accession AF135155) was amplified by LR-PCR from genomic DNA using the following primers FW 5′-GATATCTAGGCCTTGTAATATTTTTGATGAA-3′ (EcoR V linker) and RW 5′-AGATCTTTTTCACTGCTTGTTTTGGTATCTA-3′ (Bgl II linker) The amplified fragment was tailed and ligated into the pGEMT easy vector. The promoter fragment from 819 to −1 (818 bp) was excised by digesting the plasmid with a combination of EcoR V and Bgl II, the purified fragment was ligated into the corresponding sits in Bracon3. The Bracon3 construct containing 35S-CHS-E9 was previously prepared by digesting the plasmid with EcoR V and Bgl II, which released the 35s promoter the vector was gel purified. The legations replaced the 35S promoter with the promoter of CAD1 gene. The cassette pCAD1-CHS-E9 was excised by digesting with Kpn I and ligating this cassette into the Kpn I site of cam2000 FIG. 12.

### EXAMPLE 3

### Plasmid Constructions for Heavy Metal Binding

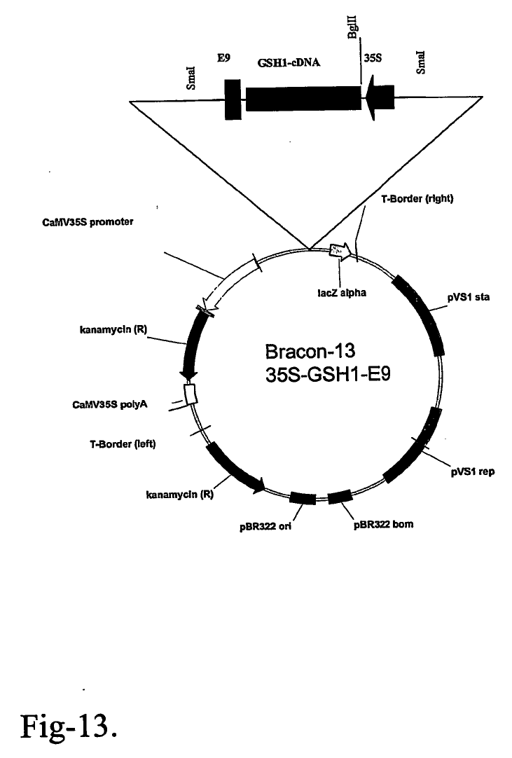

GSH-1 cDNA (Glutatmate-cysteine ligase chloroplast isoform, gene bank accession, Z29490) was amplified with the primers FW 5′-GTTAACATGGCGCTCTTGTCTCAAGCAGGAG-3′ (Hpa I linker) and RW 5′-GTTAACTTATAGACACCTTTTGTTCACGTCC-3′ (Hpa I linker) The amplified fragment was tailed and ligated into the pGEM-Teasy vector. The GSH1 cDNA was released by digestion with Hpa I, and ligated the fragment into the Stu I site in Pbs35S-E9 clonings vector. The cassette 35S-GSH1-E9 was obtained by digesting the plasmid with Sma I. The Sma I fragment was inserted into the Sma I site in the transformation vector Cam2300 FIG. 13.

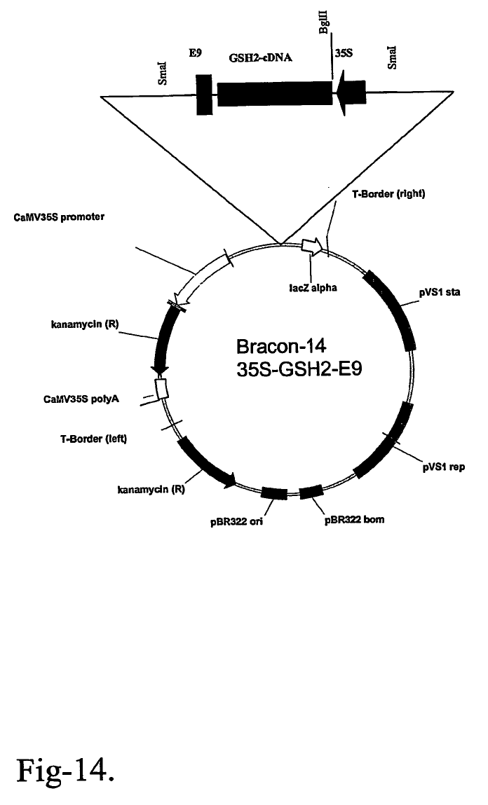

GSH-2 cDNA (Glutathione synthtase, gene bank accession X83411) was amplified by long-range PCR using the primer combination FW 5′-GTTAACATGGAATCACAGAAACCCATTTTCG-3′ (Hpa I linker) and RW 5′-GTTAACTCAATTCAGATAAATGCTGTCCAAG-3′ (Hpa I linker) on a flower cDNA library. The obtained fragment where tailed and ligated into the pGem-Teasy vector. The insert was excised by digestion of the plasmid with HpaI and the blunt end fragment inserted in the custom made vector PBS 35S-E9. The cassette 35S-GSH2-E9 was remobilised by digestion with Sma I. The Sma I fragment was ligated into the Sma I site of Cam2300 FIG. 14.

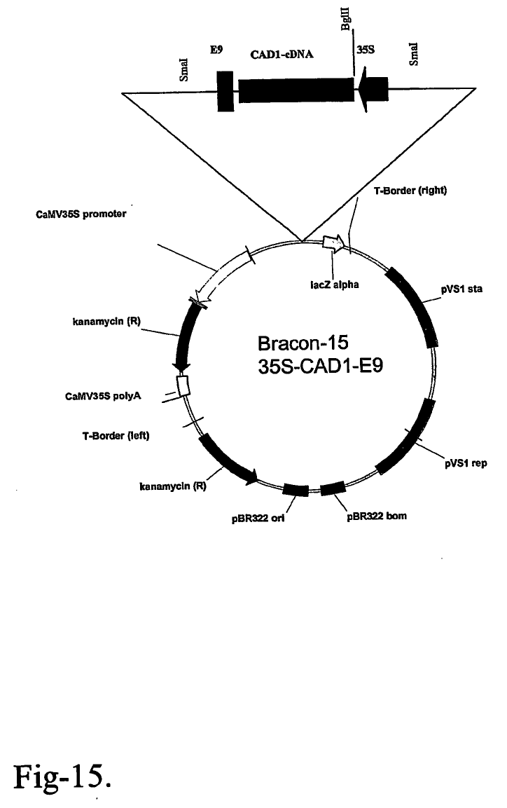

CAD-1 cDNA (Phytochelatin synthase Haet al. 1999, gene bank accession AF135155) cDNA was obtained by LR PCR using linkered primers FW 5′-GGATCCATGGCTATGGCGAGTTTATATGC-3′ (BamHI linker) and RW 5′-GCTAGCCTAATAGGCAGGAGCAGCGAGAT-3′ (NheI linker) The cDNA was amplified using a cDNA laibry produced from flowers. The resolving cDNA where tailed and cloned into the pGem-Teasy vector and subsequently sequenced to confirm the correct gene was amplified. The CAD1 cDNA was excised by EcoR I and the released fragment blunted using Mung Bean nuclease. This blunt end fragment was ligated into the Pbs 35S-E9 vector witch was pre-treated with Stu I and Cip'ed giving a dephosporylated blunt end vector. The whole cassette 35S-CAD1-E9 was realised by digestion with Sma I and transferred into the Sma I site of Cam2300 giving the construct shown in FIG. 15.

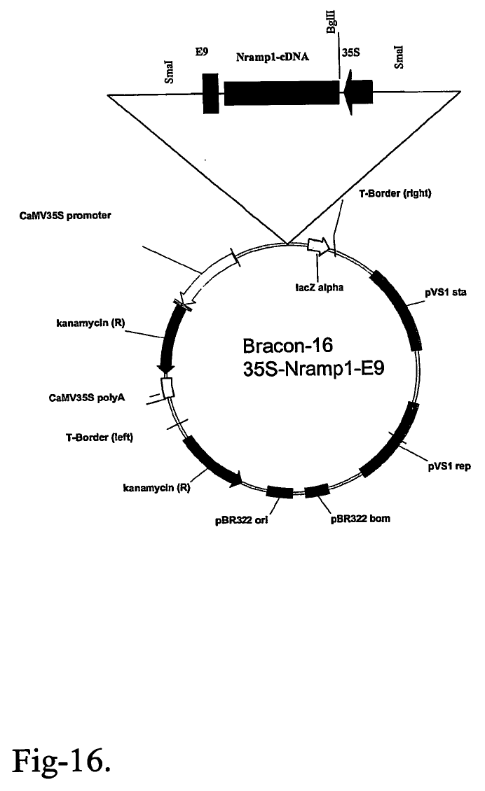

Nramp-1 cDNA (gene bank accession AF165125) was obtained by LR-PCR by the use of linkered FW 5′-AGATCTATGGCGCTACAGGATCTGGACG-3′ (Bgl II linker) and RW 5′-GCTAGCTCAGTCAACATCGGAGGTAGATA 3′ (NheI linker) the amplified product was cloned into the pGem-Teasy vector system (Promega) and sequenced. After sequencing, the cDNA was realest by digestion with Not I restriction enzyme and blunted with mung bean nuclease. This blunt end fragment was ligated into the Pbs 35S-E9 vector wich was pre-treated with Stu I and Cip'ed giving a dephosporylated blunt end vector. The cassette 35S-Nramp1-E9 was excised by Sma I and ligated into the 2300 Cambria vectors Sma I site. This construct is shown in FIG. 16.

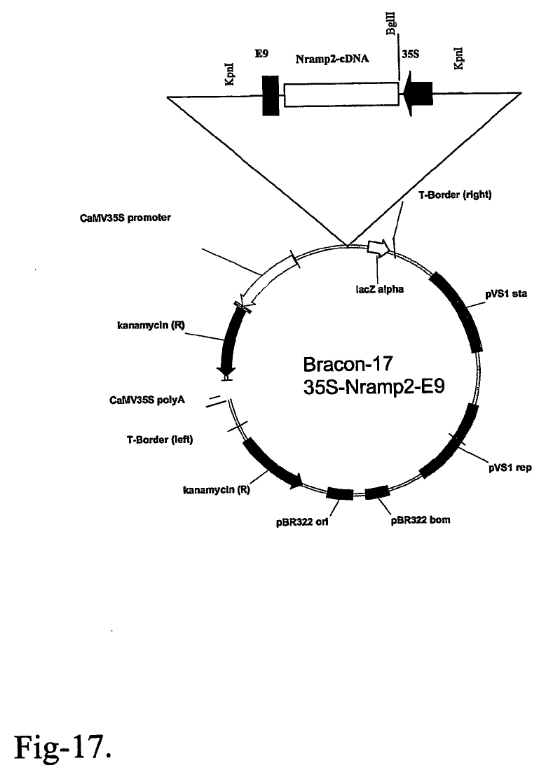

Nramp-2 cDNA (gene bank accession AF141204, Alonso et al. 1999) was obtained using same methods as descript above, by the use of FW 5′-CCATGGATGGAAAACGACGTCAAAGAGAA-3′ (NcoI linker) and RW 5′-GCTAGCCTAGCTATTGGAGACGGACACTC-3′ (NheI linker) The Nramp2 cDNA was excised from the T-Easy vector by Not I and blunted, the blunt fragment was ligated into the Stu I site of Pbs35S-E9 vector. The cassette 35S-Nramp2-E9 was excised by digestion of the vector with Kpn I. This cassette was ligated into the Kpn I site of the Cambria 2300 vector as shown in FIG. 17.

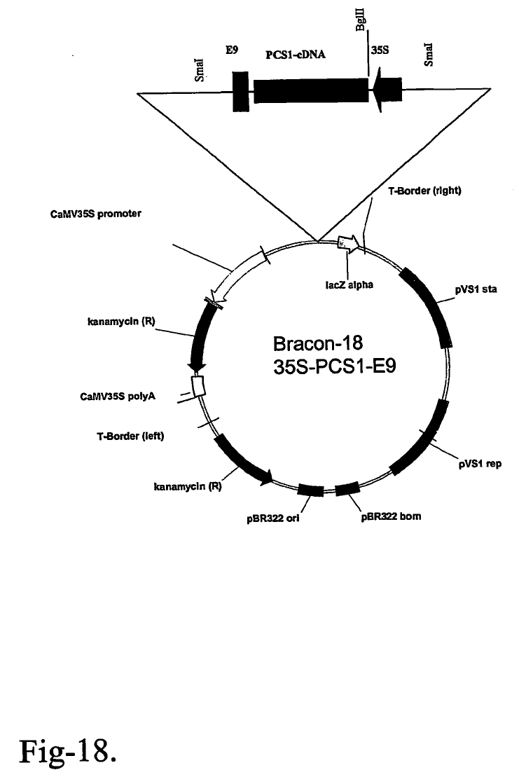

PCS-1cDNA (gene bank accession AF461180) A full length cDNA where generated by LR-PCR by the use of FW 5′-GGATCCATGGCTATGGCGAGTTTATATCG-3′ (BamH I linker) and RW 5′-GCTAGCCTAATAGGCAGGAGCAGCGAGAT-3′ (Nhe I linker). The PCR product where tailed with Taq polymerase and later ligated into pGEM-TEasy sequenced and moved into clonings vector Pbs35S-E9 by excising the fragment from pGEM-Teasy vector with EcoRI enzyme and bunting the fragment with Mung bean nuclease and ligating the fragment into the Stu I site. The cassette 35S-PCS1-E9 was released by digesting the vector with SmaI and the cassette was cloned into the SmaI site of the Cam2300 transformation vector as shown in FIG. 18.

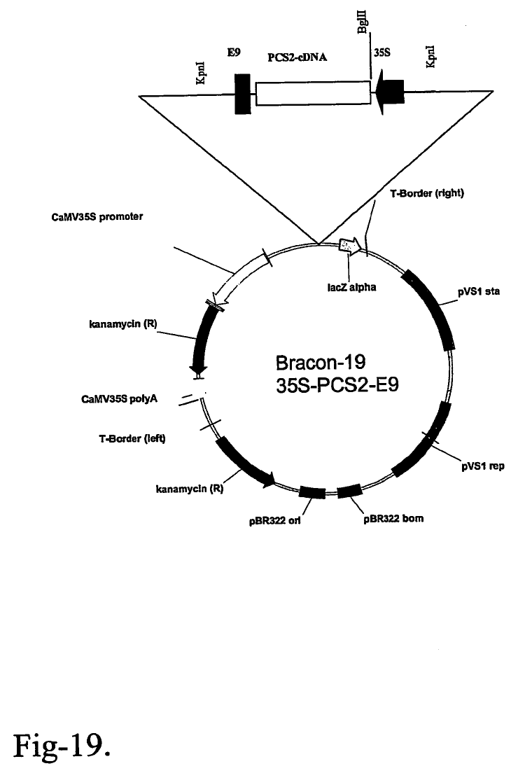

PCS-2 cDNA (gene bank accession AY044049) was amplified by LR-PCR using a combination of the FW primer 5′-GTTAACATGTCTATGGCGAGTTTGTATCGG-3′ (Hpa I linker) and RW 5′-GTTAACTTAGGCAGGAGCAGAGAGTTCTTC-3′ (Hpa I linker) the obtained fragment was tailed and ligated into the pGEM-Teasy vector. The PCS2 cDNA was released by digestion with Hpa I and the isolated fragment ligated into the Stu I site of Pbs35S-E9. The cassette 35S-PCS2-E9 was extracted by digesting the plasmid with Kpn the cassette was ligated into the Kpn I site of Cam2300 transformation vector FIG. 19.

### EXAMPLE 4

### Plasmid Constructions for Detection of Nitro-Containing Compounds

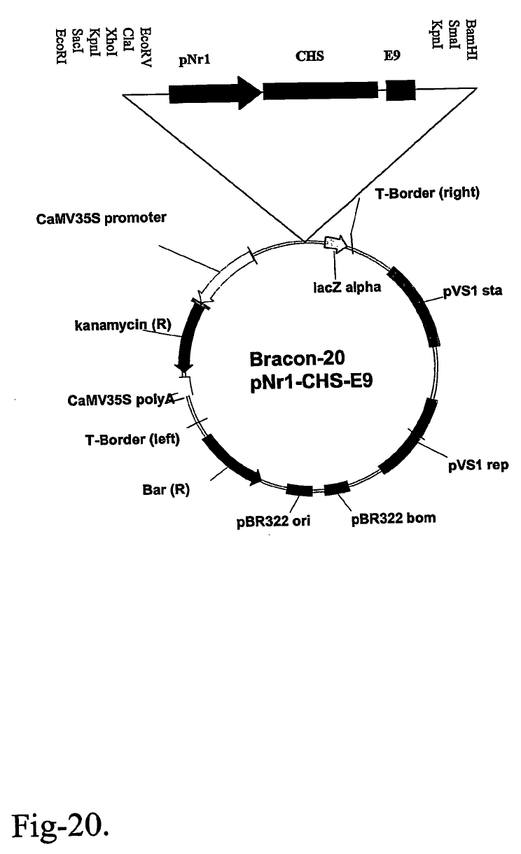

Nr-1 5′ UTR (Nitrate reductase 1, gene bank accession AC012193) was amplified using the primer combination FW 5′-GATATCCTTGAGTCATACATCTATGATA-3′ (EcoR I linker) and RW (5′ AGATCTCCATGGTTTAGTGATTGAACCGGTG-3′ (Bgll I linker). The amplified fragment (pNR1) spading the genomic sequence from −1574 to −1 giving a fragment of 1573 bp. The amplified fragment pNr1 was tailed and ligated into the pGEM-Teasy vector. The promoter fragment was released by digesting the plasmid with EcoR V/Bgl II. At the same time Plasmid of Pbs 35S-CHS-E9 (see FIG. 4.) was digested with EcoR V7Bgl II, witch releases the 35S promoter, and the vector was gel isolated and the pNr1 fragment ligated into the Pbs-CHS-E9 vector. Digesting the construct with Kpn I excised the cassette pNr1-CHS-E9. The resolving cassette fragment was ligated into the Kpn I site of cam2200, giving the construct shown in FIG. 20.

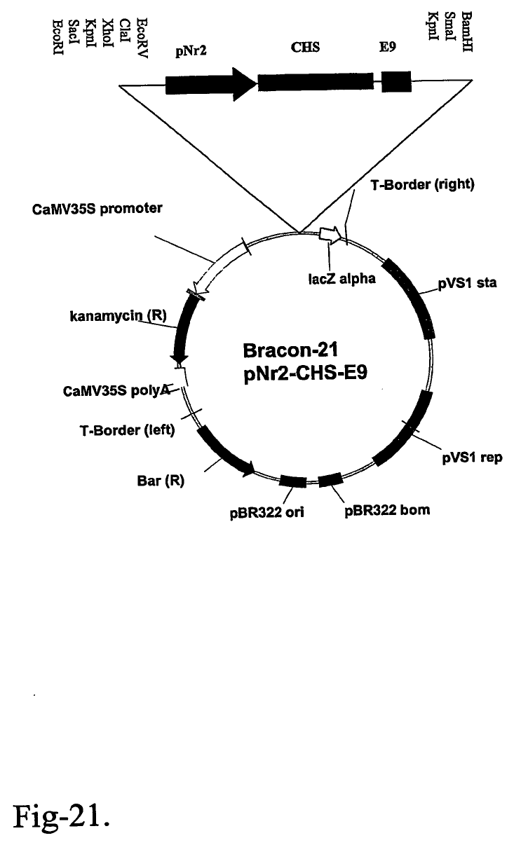

Nr-2 5′ UTR (Nitrate reductase 2, gene bank accession X13435) was amplified by LR-PCR from genomic DNA using the following primers FW 5′-GATATCGATAATTCTTTAATTTACTGG (EcoR V linker and RW 5′-GGATCCGCTAATATGTGAAAGGTTGTAC-3′ (BamH I linker) the amplified fragment was tailed and ligated into the pGEMT easy vector. The promoter fragment pNr2 from −805 to +3 was released from the pGEMT easy vector by digestion with EcoR VI BamH I. The obtained fragment was replacing the 35S promoter in the Bracon3 plasmid giving a Pbs-pNr2-CHS-E9 cassette. The cassette was excised by digestion with Kpn I and this cassette was ligated into the Kpn I site in the cam2200 transformation vector. The following construct was generated in this way FIG. 21.

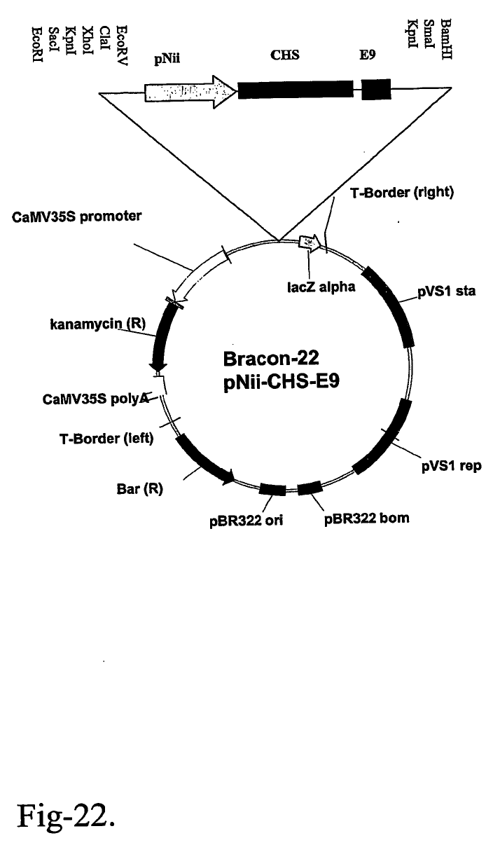

Nil 5′ UTR (Nitrite reductase gene bank accession 511655) promoter was amplified from genomic DNA using a combination of the Fw primer 5′-GTTAACCCCTAATGACCACATCAACCTTG-3′ (Hpa I linker) and RW 5′AGATCTGATGATGGCGGAAGAAGGAG (Bgl II linker). The obtained fragment spading the genomic sequence from −999 to −1 (998 bp) was tailed and ligated into the pGEMT easy vector. The pNii fragment was released by digestion with the restriction enzymes Hpa I and Bgl II. The Bracon3 plasmid was prepared for leigatin by digestion with EcoR VI Bgl II by witch the 35S promoter was removed, and the pNii promoter was ligated into the sites giving the cassette pNii-CHS-E9. The plasmid with the cassette was digested with Kpn I and the cassette ligated into the Kpn I site of the cam2200 transformation vector FIG. 22.

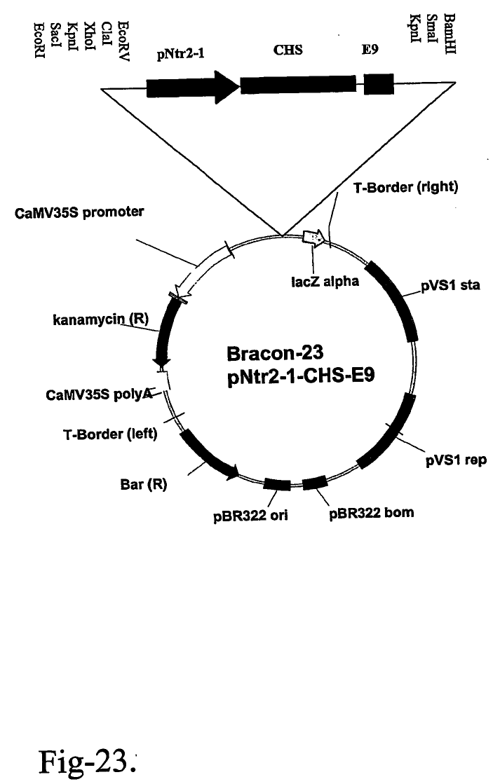

Ntr-2-1 5′UTR (High-affinity nitrate transporter ACH2 (gene bank accession AF019749) was amplified by LR-PCR from genomic DNA using the following primers FW 5′-GATATCCCAAAGCAGCAACCATTTTTCC-3′ (EcoR V linker) and RW 5′-AGATCTGTATTTTAAACGTATCAAGTTCC -3′ (Bgl II linker) the amplified fragment was tailed and ligated into the pGEMT easy vector. The promoter fragment pNtr2-1-from −974 to −1 was released from the pGEMT easy vector by digestion with EcoR VI Bgl II. The obtained fragment was replacing the 35S promoter in the Bracon3 plasmid. This was don by digesting the Bracon3 plasmid with EcoR VI Bgl II and Isolating the vector. Ligating the pNtr-2-1 fragment in the isolated vector gave the cassette Pbs-pNtr2-1-CHS-E9. The cassette was excised by digestion with Kpn I and was ligated into the Kpn I site in the cam2200 transformation vector. The following construct was generated in this way FIG. 23.

### EXAMPLE 5

### Plasmid Constructions for Reduction of Nitro-Containing Compounds

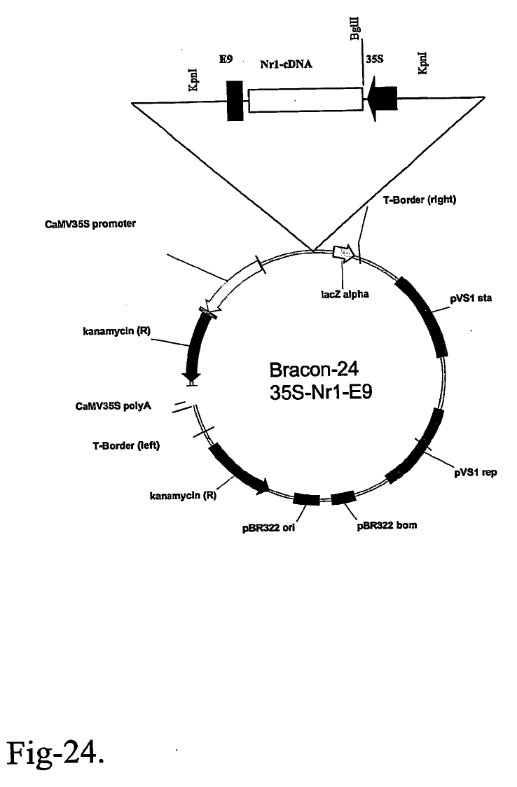

Nr-1 cDNA (Nitrate reductase 1, gene bank accession AC012193) was amplified using the primer combination FW 5′-GTTAACATGGCGACCTCCGTCGATAAC-3′ (HpaI linker) and the RW primer 5′-GTTAACCTAGAAGATTAAGAGATCCTCC-3′ (HpaI linker) the amplified fragment was tailed and ligated into the pGEM-Teasy vector. The Nr1 cDNA was released by digestion with Hpa I, and ligated into the Stu I site in Pbs35S-E9 clonings vector. The cassette 35S-Nr1-E9 was obtained by digesting the plasmid with Kpn I. The Kpn I fragment was inserted into the Kpn I site in the transformation vector Cam2300 FIG. 24.

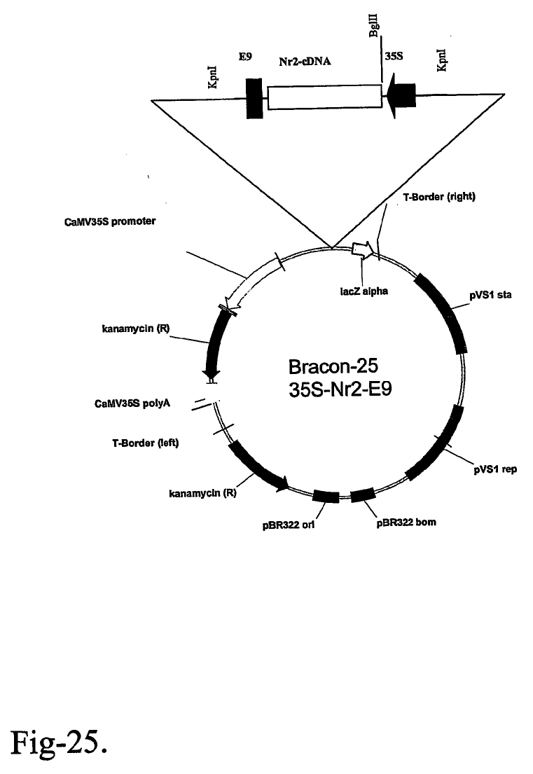

Nr-2 cDNA (Nitrate reductase 2, gene bank accession X13435) was obtained by LR-PCR using a cDNA library. As template and the FW primer 5′-GTTAACTCGGCTGACGCGCCTCCTAGTC-3′ (HpaI linker) in combination with RW primer 5′-GTTAACGAATATCAAGAAATCCTCCTTTG-3′ (HpaI linker) the amplified fragment was tailed and ligated into the pGEM-Teasy vector. The Nr2 cDNA was released by digestion with Hpa I, giving a blunt end fragment this fragment was ligated into the Stu I seit in Pbs35S-E9 cloning vector. The cassette 35S-Nr2-E9 was obtained by digesting the Pbs35S-Nr2-E9 plasmid with Kpn I. The Kpn I fragment was inserted into the Kpn I site in the transformation vector Cam2300 FIG. 25.

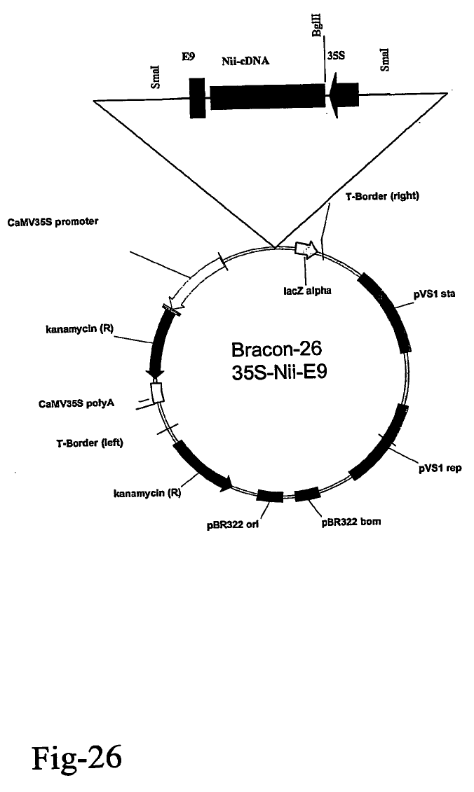

Nii cDNA The Arabidopsis thaliana nitrite reductase, gene bank accession 511655) was amplified using the FW primer 5′-GTTAACATGACTTCTTTCTCTCTCACTTTCA-3′ (HpaI linker) in combination with RW primer 5′-GTTAACTCAATCTTCATTCTCTTCTCTTTCT-3′ (HpaI linker) on a flower cDNA library. The obtained fragment where tailed and ligated into the pGem-Teasy vector. The insert was excised by digestion of the plasmid with HpaI and the blunt end fragment inserted in the custom made vector PBS 35S-E9. The cassette 35SNii-E9 was remobilised by digestion with Sma I. The Sma I fragment was ligated into the Sma I site of Cam2300 FIG. 26.

Nrt-2-1 cDNA The Arabidopsis thaliana high-affinity nitrate transporter ACH2 (gene bank accession # AF019749)

Was amplified by LR-PCR using the FW primer 5′-GTTAACATGGGTTCTACTGATGAGCCCAGAA 3′ (HpaI linker) and RW 5′-GTTAACTCAAGCATTGTTGGTTGCGTTCCCT-3′ (HpaI linker) the obtained fragment where tailed and ligated into the pGem-Teasy vector. The insert was released by digestion with HpaI and the blunt end fragment inserted in the custom made vector PBS 35S-E9. The cassette 35S-ACH2-E9 was excised using smal and transferred to the Cambria 2300 transformation vector.

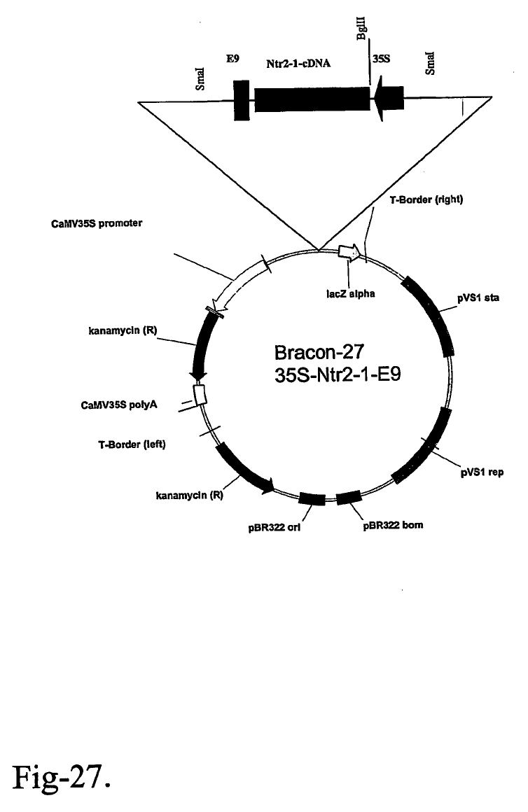

Same procedure was preformed for the following cDNAs FIG. 27.

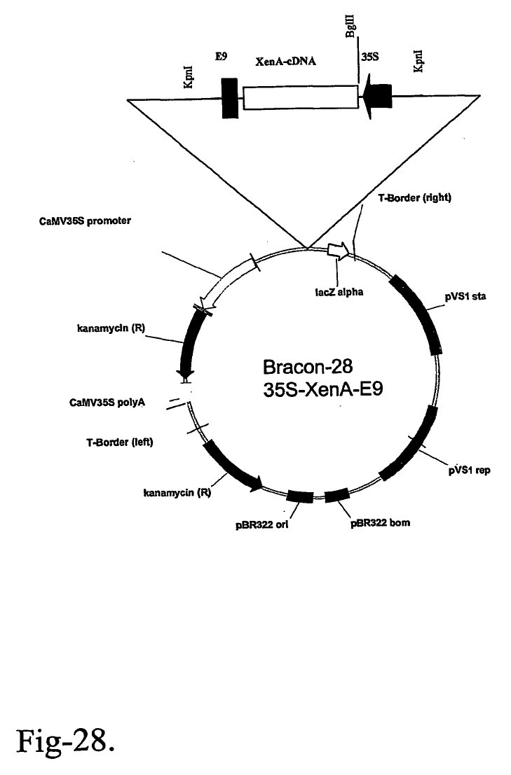

XenA cDNA (Xenobiotic reductase A, gene bank accession AF154061) was amplified with the Fw primer 5′-GTTAACATGTCCGCACTGTTCGAACCCTACA-3′ (HpaI linker) and RW 5′-GTTAACTCAGCGATAGCGCTCAAGCCAGTGC-3′ (HpaI linker) The amplified fragment was tailed and ligated into the pGEM-Teasy vector. The XenA cDNA was released by digesting the plasmid with Hpa I, giving a blunt end fragment this fragment was ligated into the Stu I site in Pbs35S-E9 cloning vector. The cassette 35S-XenA-E9 was excised by digesting the Pbs35S-XenA-E9 plasmid with Kpn I. The Kpn I fragment was inserted into the Kpn I site in the transformation vector Cam2300. FIG. 28.

XenB cDNA (Xenobiotic reductase B, gene bank accession AF154062) was amplified with the Fw primer 5′-GTTAACATGGCAATCATTTTCGATCCGATCA-3′ (HpaI linker) and RW 5′-GTTAACTTACAGCGTCGGGTAGTCGATGTAG-3′ (HpaI linker)

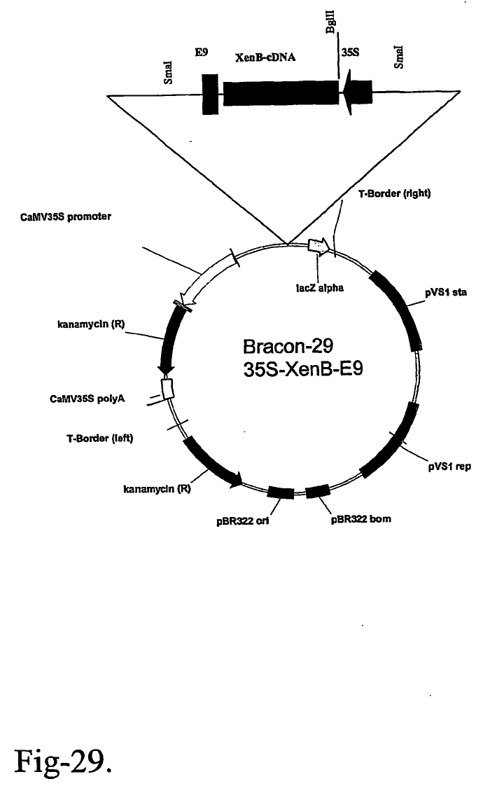

The obtained fragment where tailed and ligated into the pGem-Teasy vector. The insert was released by digestion with HpaI and the blunt end fragment inserted in the custom made vector PBS 35S-E9. The cassette 35S-XenB-E9 was excised using smal and transferred to the Cambria 2300 transformation vector. FIG. 29.

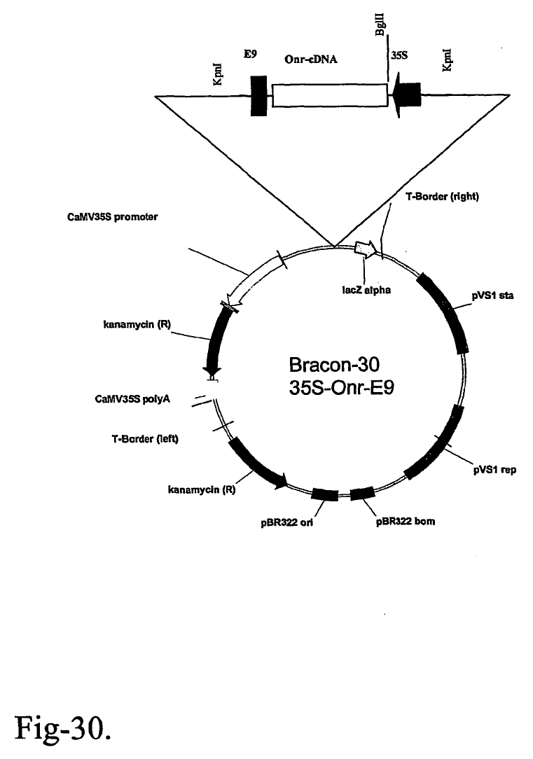

Onr cDNA (Pentaerythriol tetranitrate reductase, gene bank accession U68759) was amplified using the primer combination of FW 5′-GTTAACATGGCCGCTAAAAG-3′ (HpaI linker) and RW 5′-GTTAACGCTATCAATGTACAAGC-3′ (HpaI linker) the obtained fragment where tailed and ligated into the pGem-Teasy vector. The insert was released by digestion with Hpa I and the blunt end fragment inserted in the custom made vector PBS 35S-E9. The cassette 35S-Onr-E9 was excised using an Kpn I and transferred to the Cambria by ligating the cassette into the Kpn I site of the Cam2300 transformation vector FIG. 30.

### EXAMPLE 6

### Transformation of Plants

The following constructs were transformed into a wild type background (Bra W+ an ecotype growing in and around Copenhagen Denmark)

| | | |
|---|---|---|
| PAP1 | cDNA | 35S-PAP1-E9 |
| PAP2 | cDNA | 35S-PAP2-E9 |

The T1 lines were selected on hygromycin and red coloured plants selected. The selected lines T2 were replanted on antibiotic and plant lines segregating 1:3 for the basta marker (25% sensitive and 75% resistant plants, were propagated for future work i.e. the 1:3 indicates a single site of T-DNA integration. 12 resistant plants were transferred to soil for seed set. The seeds of T3 were replanted and plants showing 100% resistance (homozygous for the selections marker) were crossed with the tt4 mutant. In this cross the tt4×35S-PAP1-E9 F1 seeds were plated on basta and 12 bar^{r} plants transferred to soil. The segregating population from the cross displayed a distinct red or green phenotype. In the F2 generation plants showing no coloration and resistance to hygromycin were selected and propagated for seed set. Segregation analysis of the f₂ population showed a deviation from expected 3:1 ratio for the T-DNA (35S-PAP1-E9 is dominant) and 75% of the population were thus expected to be red if the tt4 mutation and the T-DNA were independent. A green:red ratio of 230:163 was observed indicating that segregating was not independent. Green Individuals of the segregating population showed both bar^{r} and bar^{s} phenotypes, proving the presence of the T-DNA in green individuals supporting the basic principle that the tt4 mutation blocks the production of pigment (anthocyanins) in these plants. The distribution of bar^{r} and bar^{s} plants in 239 green individuals from the f₂ population was 162:77. Seeds from green bar^{r} individuals showed the characteristic tt4-phenotype of the seed coat. The F3 was replanted and plants showing 100% resistance to the selection marker were finally selected. In this way plants with the following genotype were generated tt4/tt4//135S-PAP1/35S-PAP1. Same procedure was undertaken for the 35S-PAP2, leading to the final plant line tt4/tt4//35S-PAP2/35S-PAP2. The two lines were crossed, and since both plant lines were homozygotes for the tt4 mutation all progeny were tt4 mutants the dicserled line with the genotype tt4/tt4//35-PAP1/35S-PAP1//35S-PAP2/35S-PAP2 was selected by PCR using the FW 35S primer and the RW for PAP1 and PAP2. This line was named BrC line Bracifeae Cassette Line.

The following constructs were transformed into the BrC line;

Heavy Metal Detection

GSH1-CHS

GSH2-CHS

PCS1-CHS

PCS2-CHS

GST30-CHS

CAD1-CHS

Heavy Metal Binding

35S-GSH1-E9

35S-GSH2-E9

35S-CAD1-E9

35S-Nramp1-E9

35S-Nramp2-E9

35S-PCS1-E9

35S-PCS2-E9

Nitro-Detection

Nr1-CHS

Nr2-CHS

Nii-CHS

Ntr2-1-CHS

Nitro-Metabolism

35S-Nr1-E9

35S-Nr2-E9

35S-Nii-E9

35S-Nrt12-1-E9

35S-XenA-E9

35S-XenB-E9

35S-Onr-E9

The following constructs are transformed into the BraW+ line and the Col-0 line:

Heavy Metal Detection

GSH1-CHS

GSH2-CHS

PCS1-CHS

PCS2-CHS

GST30-CHS

CAD1-CHS

GSH1-LUC

GSH2-LUC

PCS1-LUC

PCS2-LUC

GST30-LUC

CAD1-LUC

GSH1-GFP

GSH2-GFP

PCS1-GFP

PCS2-GFP

GST30-GFP

CAD1-GFP

Heavy Metal Binding

35S-GSH1-E9

35S-GSH2-E9

35S-CAD1-E9

35S-Nramp1-E9

35S-Nramp2-E9

35S-PCS1-E9

35S-PCS2-E9

Nitro-Detection

Nr1-CHS

Nr2-CHS

Nii-CHS

Ntr2-1-CHS

Nr1-LUC

Nr2-LUC

Nii-LUC

Ntr2-1-LUC

Nr1-GFP

Nr2-GFP

Nii-GFP

Ntr2-1-GFP

Nitro-Metabolism

35S-Nr1-E9

35S-Nr2-E9

35S-Nii-E9

35S-Nrt12-1-E9

35S-XenA-E9

35S-XenB-E9

35S-Onr-E9

### EXAMPLE 7

### Test of the Heavy Metal Detection System in Plants

The following constructs are transformed into the BrC line

GSH1-CHS

GSH2-CHS

PCS1-CHS

PCS2-CHS

GST30-CHS

CAD1-CHS

The obtained transformed lines are tested on MS plates containing increasing amounts of the following heavy metals Cu, Zn, Cd, Hg, Pb, Co, Cr, Ni, As, Be, Se, Au, Ag. in concentrations ranging from 0.00025, 0.0005, 0.001, 0.0015, 0.002, 0.0025, 0.003, 0.004, 0.005, 0.006, 0.007, 0.008, 0.009, 0.01, 0.02, 0.03, 0.04, 0.05, 0.06, 0.07, 0.08, 0.09, e.g. 0.1, 0.2, 0.3, 0.4, 0.5, 0.6, e.g. 0.7, 0.8, 0.9, 1, e.g. 2, 3, 4, 5, 6, 7, 8, 9, 10 mM. In this way we are selecting lines which change colour at different concentrations of heavy metals. And at the same time investigating the response from different promoters to the range of heavy metals i.e. the specificity of the individual promoters. At the same time a pot experiment is being conducted 9 inch. pots with soil these pots are watered with solutions of heavy metals ranging in concentration and type identical to the plate experiment described above.

### EXAMPLE 8

### Test of the Nitro Detection System in Plants

The following constructs are transformed into the BrC line

Nr1-HS

Nr2-CHS

Nii-CHS

Ntr2-1-CHS

The obtained transformed lines are tested for the capability to develop a colour change on MS plates containing increasing amounts of the following nitro-compounds: TNT (2,4,6-trinitrotouluene), PETN (pentaerythiol tetranitrate) or RDX (Cyclotrimethylenetrinitramine), in concentrations ranging from 0.00025, 0.0005, 0.001, 0.0015, 0.002, 0.0025, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 2, 3, 4, 5, 6, 7, 8, 9, 10 mM. and lines are selected based on the observed colour change at different concentrations. A similar experiment is being conducted with plants growing in 9 inch. pots with soil in order to determine the buffer effect in soil.

### EXAMPLE 8a

The BrC line was transformed with the NII-CHS E9 construct. The NII-CHS-E9 (Ti) plant line was grown on MS plates supplemented with 0.01 mM TNT. Plants developed a distinct red pigmentation. After 2 weeks the plants were transferred to soil without TNT, where the pigmentation gradually decreased.

### EXAMPLE 9

### Test of Heavy Metal Binding

In order to enhance the capability to accumulate heavy metals the following constructs are transformed into the BrC line:

35S-GSH1-E9

35S-GSH2-E9

35S-CAD1-E9

35S-Nramp1-E9

35S-Nramp2-E9

35S-PCS1-E9

35S-PCS2-E9

Transformed lines carrying the heavy metal binding constructs are tested for the ability to increase the concentration of heavy metal in the aerial parts of the plant Seeds are spread on MS containing increasing amounts of the following heavy metals Cu, Zn, Cd, Hg, Pb, Co, Cr, Ni, As, Be, Se, Au, Ag in concentrations ranging from 0.000, 0.00025, 0.0005, 0.001, 0.0015, 0.002, 0.0025, 0.003, 0.004, 0.005, 0.006, 0.007, 0.008, 0.009, 0.01, 0.02, 0.03, 0.04, 0.05, 0.06, 0.07, 0.08, 0.09, e.g. 0.1, 0.2, 0.3, 0.4, 0.5, 0.6, e.g. 0.7, 0.8, 0.9, 1, e.g. 2, 3, 4, 5, 6, 7, 8, 9, 10 mM. Samples are analysed by standard methods for heavy metal analysis. Lines showing high, medium and low binding are selected for the crosses with heavy metal detection plants.

### EXAMPLE 10

### Test of Nitro-Metabolism

The following constructs are transformed into the BrC line:

35S-Nr1-E9

35S-Nr2-E9

35S-Nii-E9

35S-Nrt12-1-E9

35S-XenA-E9

35S-XenB-E9

35S-Onr-E9

The obtained transformed lines are tested on MS plates containing increasing amounts of the following nitro-compounds: TNT (2,4,6-trinitrotouluene), PETN (pentaerythiol tetranitrate) or RDX (Cyclotrimethylenetrinitramine), in concentrations ranging from 0.00025, 0.0005, 0.001, 0.0015, 0.002, 0.0025, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 2, 3, 4, 5, 6, 7, 8, 9, 10 mM. and plants showing more/or less resistance toward the explosives are selected for further analysis and crossing with nitro-detection lines.

### EXAMPLE 11

### Crossing of Plants to Obtain Heavy Metal Detection and Binding

A line showing higher contents of heavy metal was crossed into the detection lines, the following crosses are generated

GSH1-CHS/35S-GSH1-E9

GSH1-CHS/35S-GSH1-E9/35S-GSH2-E9

GSH1-CHS/35S-GSH1-E9/35S-GSH2-E9/35S-CAD1-E9

GSH1-CHS/35S-GSH1-E9/35S-GSH2-E9/35S-CAD1-E9/35S-Nramp1-E9

GSH1-CHS/35S-GSH1-E9/35S-GSH2-E9/35S-CAD1-E9/35S-Nramp1-E9/35S-Nramp2-E9

GSH1-CHS/35S-GSH1-E935S-GSH2-E9/35S-CAD1-E9/35S-Nramp1-E9/35S-Nramp2-E9/35S-PCS1-E9

GSH1-CHS/35S-GSH1-E9/35S-GSH2-E9/35S-CAD1-E9/35S-Nramp1-E9/35S-Nramp2-E9/35S-PGS1-E9/35S-PCS2-E9

GSH2-CHS/35S-GSH1-E9

GSH2-CHS/35S-GSH1-E9/35S-GSH2-E9

GSH2-CHS/35S-GSH1-E9/35S-GSH2-E9/35S-CAD1-E9

GSH2-CHS9/35S-GSH1-E9/35S-GSH2-E9/35S-CAD1-E9/35S-Nramp1-E9

GSH2-CHS9/35S-GSH1-E9/35S-GSH2-E9/35S-CAD1-E9/35S-Nramp1-E9/35S-Nramp2-E9

GSH2-CHS/35S-GSH1-E9/35S-GSH2-E9/35S-CAD1-E9/35S-Nramp1-E9/35S-Nramp2-E9/35S-PCS1-E9

GSH2-CHS9/35S-GSH1-E9/35S-GSH2-E9/35S-CAD1-E9/35S-Nramp1-E9/35S-Nramp2-E9/35S-PCS1-E9/35S-PCS2-E9

PCS1-CHS/35S-GSH1-E9

PCS1-CHS/35S-GSH1-E9/35S-GSH2-E9

PCS1-CHS/35S-GSH1-E9/35S-GSH2-E9/35S-CAD1-E9

PCS1-CHS9/35S-GSH1-E9/35S-GSH2-E9/35S-CAD1-E9/35S-Nramp1-E9

PCS1-CHS9/35S-GSH1-E9/35S-GSH2-E9/35S-CAD1-E9/35S-Nramp1-E9/35S-Nramp2-E9

PCS1-CHS/35S-GSH1-E9/35S-GSH2-E9/35S-CAD1-E9/35S-Nramp1-E9/35S-Nramp2-E9/35S-PCS1-E9

PCS1-CHS9/35S-GSH1-E9/35S-GSH2-E9/35S-CAD1-E9/35S-Nramp1-E9/35S-Nramp2-E9/35S-PCS1-E9/35S-PCS2-E9

PCS2-CHS/35S-GSH1-E9

PCS2-CHS/35S-GSH1-E9/35S-GSH2-E9

PCS2-CHS/35S-GSH1-E9/35S-GSH2E9/35S-CAD1-E9

PCS2-CHS/35S-GSH1-E9/35S-GSH2-E9/35S-CAD1-E9/35S-Nramp1-E9

PCS2-CHS/35S-GSH1-E9/35S-GSH2-E9/35S-CAD1-E9/35S-Nramp1-E9/35S-Nramp2-E9

PCS2-CHS/35S-GSH1-E9/35S-GSH2-E9/35S-CAD1-E9/35S-Nramp1-E9/35S-Nramp2-E9

PCS2-CHS/35S-GSH1-E9/35S-GSH2-E9/35S-CAD1-E9/35S-Nramp1-E9/35S-Nramp2-E9/35S-PCS1-E9

PCS2-CHS/35S-GSH1-E9/35S-GSH2-E9/35S-CAD1-E9/35S-Nramp1-E9/35S-Nramp2-E9/35S-PCS1-E9/35S-PCS2-E9

GST30-CHS/35S-GSH1-E9

GST30-CHS/35S-GSH1-E9/35S-GSH2-E9

GST30-CHS/35S-GSH1-E9/35S-GSH2E9/35S-CAD1-E9

GST30-CHS/35S-GSH1-E9/35S-GSH2-E9/35S-CAD1-E9/35S-Nramp1-E9

GST30-CHS/35S-GSH1-E9/35S-GSH2-E9/35S-CAD1-E9/35S-Nramp1-E9/35S-Nramp2-E9

GST30-CHS/35S-GSH1-E9/35S-GSH2-E9/35S-CAD1-E9/35S-Nramp1-E9/35S-Nramp2-E9/35S-PCS1-E9

GST30-CHS/35S-GSH1-E9/35S-GSH2-E9/35S-CAD1-E9/35S-Nramp1-E9/35S-Nramp2-E9/35S-PCS1-E9/35S-PCS2-E9

CAD1-CHS/35S-GSH1-E9

CAD1-CHS/35S-GSH1-E9/35S-GSH2-E9

CAD1-CHS/35S-GSH1-E9/35S-GSH2E9/35S-CAD1-E9

CAD1-CHS/35S-GSH1-E9/35S-GSH2-E9/35S-CAD1-E9/35S-Nramp1-E9

CAD1-CHS/35S-GSH1-E9/35S-GSH2-E9/35S-CAD1-E9/35S-Nramp1-E9/35S-Nramp2-E9

CAD1-CHS/35S-GSH1-E9/35S-GSH2-E9/35S-CAD1-E9/35S-Nramp1-E9/35S-Nramp2-E9/35S-PCS1-E9

CAD1-CHS/35S-GSH1-E9/35S-GSH2-E9/35S-CAD1-E9/35S-Nramp1-E9/35S-Nramp2-E9/35S-PCS1-E9/35S-PCS2-E9

### EXAMPLE 12

### Crossing of Plants to Obtain Increased NO2 Release

In order to increase the release NO₂ from the explosives, the following crosses are generated:

Nr1-CHS/35S-Nr1-E9

Nr1-CHS/35-Nr1-E9/35S-Nr2-E9

Nr1-CHS/35S-Nr1-E9/35S-Nr2-Nii-E9

Nr1-CHS/35S-Nr1-E9/35S-Nr2-E9/35S-Nii-E9/35-Nrt12-1-E9

Nr1-CHS/35S-Nr1-E9/35S-Nr2-E9/35S-Nii-E9/35S-Nrt12-1-E9/35S-XenA-E9

Nr1-CHS/35Nr1-E9/35S-Nr2-E9/35S-Nii-E9/35S-Nrt12-1-E9/35S-XenA-E9/35S-XenB-E9

Nr1-CHS/35S-Nr1-E9/35S-Nr2-E9/35S-Nii-E9/35S-Nrt12-1-E9/35XenA-E9/35S-XenB-E9/35S-Onr-E9

Nr2-CHS/35S-Nr1-E9

Nr2-CHS/35S-Nr1-E9/35S-Nr2-E9

Nr2CHS/35S-Nr1-E9/35S-Nr2-E9/35S-Nii-E9

Nr2-CHS/35S-Nr1-E9/35-Nr2-E9/35S-Nii-E9/35S-Nrt12-1-E9

Nt2-CHS/35S-Nr1-E9/35S-Nr2-E9/35S-Nii-E9/35S-Nrt12-1-E9/35S-XenA-E9

Nr2-CHS/35Nr1-E9/35S-Nr2-E9/35S-Nii-E9/35S-Nrt12-1-E9/35S-XenA-E9/35S-XenB-E9

Nr2-CHS/35S-Nr1-E9/35S-Nr2-E9/35S-Nii-E9/35S-Nrt12-1-E9/35XenA-E9/35S-XenB-E9/35S-Onr-E9

Nii-CHS/35S-Nr1-E9

Nii-CHS/35-Nr1-E9/35S-Nr2-E9

Nii-CHS/35S-Nr1-E9/35S-Nr2-Nii-E9

Nii-CHS/35S-Nr1-E9/35S-Nr2-E9/35S-Nii-E9/35-Nrt12-1-E9

Nii-CHS/35S-Nr1-E9/35S-Nr2-E9/35S-Nii-E9/35S-Nrt12-1-E9/35S-XenA-E9

Nii-CHS/35Nr1-E9/35S-Nr2-E9/35S-Nii-E9/35S-Nrt12-1-E9/35S-XenA-E9/35S-XenB-E9

Nii-CHS/35S-Nr1-E9/35S-Nr2-E9/35S-Nii-E9/35S-Nrt12-1-E9/35XenA-E9/35S-XenB-E9/35S-Onr-E9

Ntr1-2-CHS/35S-Nr1-E9

Ntr1-2-CHS/35-Nr1-E9/35S-Nr2-E9

Ntr1-2-CHS/35S-Nr1-E9/35S-Nr2-Nii-E9

Ntr1-2-CHS/35S-Nr1-E9/35S-Nr2-E9/35S-Nii-E9/35-Nrt12-1-E9

Ntr1-2-CHS/35S-Nr1-E9/35S-Nr2-E9/35S-Nii-E9/35S-Nrt12-1-E9/35S-XenA-E9

Ntr1-2-CHS/35Nr1-E9/35S-Nr2-E9/35S-Nii-E9/35S-Nrt12-1-E9/35S-XenA-E9/35S-XenB-E9

Ntr1-2-CHS/35S-Nr1-E9/35S-Nr2-E9/35S-Nii-E9/35S-Nrt12-1-E9/35XenA-E9/35S-XenB-E9/35S-Onr-E9

### EXAMPLE 13

### Regulation of Heavy Metal Promoters

In order to get a more detailed description of the promoter-LUC lines

GSH1-LUC

GSH2-LUC

PCS1-LUC

PCS2-LUC

GST30-LUC

CAD1-LUC

are generated in the wild type BraW+ and Col-0 was plated on MS plates containing the following heavy metals; con, Cu++, Ni++, Zn++, Ag++ Hg++, Cd++ and Pb++ and imaged with a N2 cooled CCD camera as described in Meier et al. 2000. Lines showing clear induction after heavy metal treatment were tested for specificity to the individual metals.

### EXAMPLE 13a

The GSH1-LUC-E9 construct was transformed into the BrC line. Treatment of leaves of (t2) plants treated for 30 min with either H2O, 100 μM Cd2+ or 100 μM Cu2+ showed that both heavy metals gave induction of the promoter after 30 minutes as could be assessed by Imaging with a N2 cooled CCD camera. It was demonstrated that a related species, Capsella Bursa-pastoris, could also be transformed with a GSH1-promoter construct (GSH1-GFP) by selecting transformed plants on hygromycin plates.

### EXAMPLE 14

### Expression Pattern of Heavy Metal Promoters

In order to get the expression pattern of the promoter lines in the BraW+ and Col-0 background carrying the following constructs

GSH1-GFP

GSH2-GFP

PCS1-GFP

PCS2-GFP

GST30-GFP

CAD1-GFP

are analysed by confocual microscopy in order to elute the expression pattern of the promoters.

### EXAMPLE 15

### Regulation of Nitro-Promoters

In order to get a more detailed description of the regulation of the nitro-promoter-LUC lines

Nr1-LUC

Nr2-LUC

Nii-LUC

Ntr2-1-LUC

are generated in the wild type BraW+ and Col-0. Seed were plated on MS plates containing the following explosives TNT (2,4,6-trinitrotouluene), PETN (pentaerythiol tetranitrate) or RDX (Cyclotrimethylenetrinitramine). The concentrations for the different explosives was 0.01 μM, 0.02 μM, 0.03 μM, 0.04 μM, 0.05 μM, respectfully The plates where imaged with a N2 cooled CCD camera 10 days after plating.

### EXAMPLE 15a

The BrC line was transformed with the NII-LUC-E9 construct.

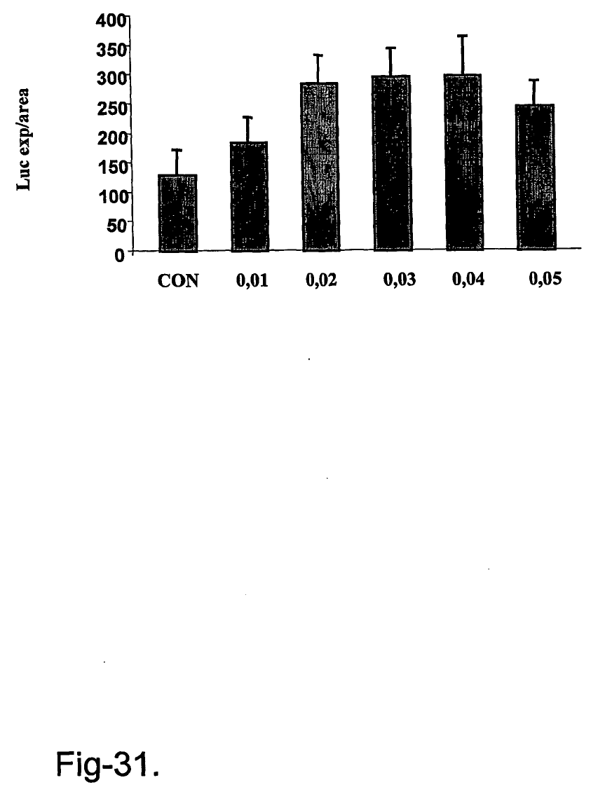

The plants transformed with the NII-LUC-E9 construct were grown on MS plates supplemented with increasing concentrations (0.01 μM-0.05 μM) of TNT (2,4,6-trinitrotoluen). At high concentrations the plants showed retarded growth. The bar diagram shown in FIG. 31 gives the LUC expression/area values for the different treatments showing an Induction of the promoter.

### EXAMPLE 16

### Expression Pattern of Nitro-Promoters

In order to get the expression pattern of the promotor lines in the BraW+ and Col-0 background carrying the following constructs

Nr1-GFP

Nr2-GFP

Nii-GFP

Ntr2-1-GFP

are analysed by confocual microscopy i.e. order to elute the expression pattern of the promoters.

### EXAMPLE 17

Bacterial cells of E. Coli (C), Pseudomonas putita (PU), Pseudomonas sydngae (SY), Pseudomonas fluorescens (FL) were grown on LB plates with increasing concentrations of TNT and RDX. The PU and FL show more resistance towards the explosives indicating the presence of the reductases ExenA and ExenB. These were subsequently cloned and used for plant transformations.

### References

Alonso, J. M., Hirayama, T., Roman G., Nourizadeh, S, Ecker J. R.

EIN2, a bifunctional transducer of ethylene and stress responses in Arabidopsis. Science 1999 June 25;284(5423):2148-52
Bechtold, N. and Pelletier, G.

In planta Agrobacterium-mediated transformation of adult Arabidopsis thaliana plants by vacuum infiltration. Methods Mol Biol 82, 259-66 (1998)
Boline F.

Leveling land mines with biotechnology. Nat Biotechnol. 1999 August;17(8):732
Borevitz, J. O., Xia, Y., Blount, J., Dixon, R. A., Lamb, C.

Activation tagging identifies a conserved MYB regulator of phenylpropanoid biosynthesis. Plant Cell 2000 December;12(12):2383-2394
Borgwardt, C.

ODIS—Ordnance Detection and Identification System. In Proceedings of WAPM'95, Lausanne, Switzerland, Jun. 30-Jul. 1st, 1995, pp. 37-43.
Court, D. L., Sawitzke, J. A. and Thomason, L. C.

Genetic engineering using homologous recombination. Annu Rev Genet 36, 361-688 (2002)
Cremers, D. A., Ebinger, M. H., Breshears D. D., Unkefer, P. J., Kammerdiener, S. A., Ferris, M. J., Catlett, K. M. and Brown, J. R.

Measuring Total Soil Carbon with Laser-induced Breakdown Spectroscopy (LIBS) Journal of Environmental Quality 30:2202-2206 (2001)
Gallie, D. R., Sleat, D. E., Watts, J. W., Turner, P. C., and Wilson, T. M.

The 5′-leader sequence of tobacco mosaic virus RNA enhances the expression of foreign gene transcripts in vitro and in vivo. Nucleic Acids Res 15, 3257-73 (1987)
Gong, P., Wilke, B., Fleischmann, S.

Soil-based phytotoxicity of 2,4,6-trinitrotoluene (TNT) to terrestrial higher plants. Arch Environ Contam Toxicol. 1999 February;36(2):152-7
Gorontzy, T., Drzyzga, O., Kahl, M. W., Bruns-Nagel, D., Breitung, J., von Loew, E., and Blotevogel, K. H.

Microbial degradation of explosives and related compounds. Crit. Rev. Microbiol. 20:265-284 (1994)
Ha, S. B., Smith, A. P., Howden, R., Dietrich, W. M., Bugg, S., O'Connell, M. J., Goldsbrough, P. B., Cobbett, C. S.

Phytochelatin synthase genes from Arabidopsis and the yeast Schizosaccharomyces pombe. Plant Cell 1999 June;11(6):1153-64
Haas, R. J., and Simmons, B. P.

Measurement of Trinitrotoluene (TNT) and Hexahydro-1,3,5-trinitro-1,3,5-triazine (RDX) in Soil by Enzyme Immunoassay and High Performance Liquid Chromatography (EPA Method 8330). California Environmental Protection Agency, Department of Toxic Substances Control, Hazardous Materials Laboratory, March. (1995)
He, M., Yang, Z Y., Nie, Y. F., Wang, J., Xu, P.

New type of class I bacterial 5-enopyrvylshikimate-3-phosphate synthase mutants with enhanced tolerance to glyphosate. Biochim Biophys Acta 2001 Nov. 7;1568(1):1-6.
Hesselsoe, M., Jensen, D., Skals, K., Olesen, T., Moldrup, P., Roslev, P., Mortensen, G. K., Henriksen.

Degradation of 4-nonylphenol in homogeneous and nonhomogeneous mixtures of soil and sewage sludge. Environ Sci Technol 2001 Sep. 15;35(18):3695-700.
Jefferson, R. A., Kavanagh, T. A., Bevan, M. W.

GUS fusions: beta-glucuronidase as a sensitive and versatile gene fusion marker in higher plants. EMBO J. 1987 Dec. 20;6(13):3901-7.
Jefferson, R. A., Bevan, M., Kavanagh, T.

The use of the Escherichia coli beta-glucuronidase as a gene fusion marker for studies of gene expression in higher plants. Biochem Soc Trans. 1987 February;15(1):17-8.
Kertbundit, S., De Greve, H., Deboeck, F., Van Montagu, M., Hernalsteens, J. P.

In vivo random beta-glucuronidase gene fusions in Arabidopsis thaliana. Proc Natl Acad Sci USA. 1991 Jun. 15;88(12):5212-6.
Lamb, C. J., Ryals, J. A., Ward, E. R., Dixon, R. A.

Emerging strategies for enhancing crop resistance to microbial pathogens. Biotechnology (NY) 1992 November;10(11):1436-45
May, M. J., Leaver, C. J.

Arabidopsis thaliana gamma-glutamylcysteine synthetase is structurally unrelated to mammalian, yeast, and Escherichia coli homologs. Proc Natl Acad Sci USA 1994 Oct. 11;91 (21):10059-63
Meier, C., Bouquin, T., Nielsen, M. E., Raventos, D., Maftsson, O., Rocher, A., Schomburg, F., Amasino, R. M., Mundy, J.

Gibberellin response mutants identified by luciferase imaging. Plant J 2001 March; 25(5):509-19
Miljøtilstandsrapport: “Natur og miljø 1997. P\dot{a}virkninger og tilstand”, Danmarks Miljøundersøgelser (DMU) (National Environmental Research Institute, Denmark)
Pietrzak, M., Shillito, R. D., Hohn, T., Potrykus, I.

Expression in plants of two bacterial antibiotic resistance genes after protoplast transformation with a new plant expression vector. Nucleic Acids Res 1986 Jul. 25;14(14):5857-68
Raines, G. L., and Canney, F. C.

Vegetation and geology in Siegal, B. S., and Gillespie, A. R., eds., Remote Sensing in Geology: New York, John Wiley and Sons, Inc., p. 365-380 (1980)
Rugh, C. L., Wilde, H. D., Stack, N. C., Thompson, D. M., Summers, A. O. and Meagher, R. B.

Mercuric ion reduction and resistance in transgenic Arabidopsis thaliana plants expressing a modified bacterial merA gene. 1996. Proceedings, National Academy of Sciences, 93:3182-3187.
Sambrook et al. 1989

Molecular cloning a laboratory manual second edition cold spring harbor laboratory press 1989
Scudiero et al. 2001

Structural and function analysis of metal regulatory elements in the promotor region of genes encoding metallothionein isoforms in the Antarctic fish Chionodraco hamatus (icefish) Gene (274) 199-208.
Shen, C. F., Gulot, S. R., Thiboutot, S., Ampleman, G., Hawari, J.

Fate of explosives and their metabolites in bioslurry treatment process. Biodegradation 1998; 8: 339-347.
Spain, J. C.

Biodegradation of nitroaromatic compounds. Annu. Rev. Microbiol. 49:523-555 (1995)
Taha, M. R., Soewarto, H., Acar, Y. B., Gale, R. J., Zappi, M. E.

Surfactant enhanced desorption of TNT from soil. Water, Air and Soil pollution 1997; 100: 33-48
Velten, J., Schell, J.

Selection-expression plasmid vectors for use in genetic transformation of higher plants. Nucleic Acids Res 1985 Oct. 11;13(19):6981-98
White, G. F. and Snape, J. R.

Microbial cleavage of nitrate esters: defusing the environment. J. Gen. Microbiol. 139:1947-1957 (1993)
Zhu, Y. L., Pilon-Smits, E. A., Tarun, A. S., Weber, S. U., Jouanin, L., Terry, N.

Cadmium tolerance and accumulation in Indian mustard is enhanced by overexpressing gamma-glutamylcysteine synthetase. Plant Physiol. 1999 December;121(4):1169-78

1. A reporter system giving rise to a directly monitorable phenotypic trait in a plant in the presence of solely an outer stimulus, said reporter system comprising a gene which is not part of the natural plant genome encoding a product which is involved in the development of said directly monitorable phenotypic trait in response to the presence of said outer stimulus.

2. A reporter system according to claim 1, wherein said directly monitorable phenotypic trait is a result of altered expression of said gene in response to the presence of the outer stimulus.

3. A reporter system according to claim 2, wherein a sensor system brings about said altered gene expression in response to the presence of the outer stimulus.

4. A reporter system according to claim 3, wherein the sensor system comprises a regulatory element.

5. A reporter system according to claim 4, wherein the regulatory element comprises a metal response element (MRE) with a sequence selected from the group consisting of TGCACCC, TGCACGC, TGCACAC and TGCGCAC.

6. A reporter system according to claim 3, wherein the sensor system comprises a promoter, the activity of said promoter being affected by the presence of the outer stimulus.

7. A reporter system according to claim 6, wherein said promoter is operatively coupled to the gene.

8. A reporter system according to claim 6, wherein the promoter is selected from the group consisting of Arabidopsis thaliana gamma-glutamylcysteine synthetase (X80377. X81973 and X84097), Arabidopsis thaliana phytochelatin synthase (PCS1, AF093753), Arabidopsis thaliana IRT1, and IRT2 metal transporters (U27590 and T04324), Arabidopsis thaliana AtPCS1, and AtPCS2 (W43439 and AC003027)

9. A reporter system according to claim 1, wherein the gene or genes are involved in the production of a visible colour change in plants.

10. A reporter system according to claim 1, wherein the gene or genes is involved in the phenylpropanoid metabolism.

11. A reporter system according to claim 1, wherein the gene or genes is involved in the biosynthesis of pigment.

12. A reporter system according to claim 1, wherein the gene or genes is involved in the biosynthesis of flavonoids.

13. A reporter system according to claim 1, wherein the gene or genes are involved in the biosynthesis of anthocyanins.

14. A reporter system according to claim 1, wherein the gene is chalcone synthase (CHS).

15. A reporter system according to claim 1, wherein the gene is chalcone isomerase (CHI).

16. A reporter system according to claim 1, wherein the gene is dihydroflavonol reductase (DFR).

17. A reporter system according to claim 1, wherein any endogenous copies of said gene or genes are non-functional.

18. A reporter system according to claim 17, wherein the endogenous gene or genes are involved in the production of pigment.

19. A reporter system according to claim 17, wherein the endogenous gene or genes i are involved in the flavonoid biosynthesis pathway.

20. A reporter system according to claim 17, wherein the endogenous gene or genes are involved in the synthesis of an agent selected from the group consisting of tetrahydroxychalcon and chalcone.

21. A reporter system according to claim 17, wherein the endogenous gene is the CHS gene (tt4 mutant).

22. A reporter system according to claim 17, wherein the endogenous gene or genes are involved in the formation of 2S-flavanones, narringenein or ligquritigenin.

23. A reporter system according to claim 17, wherein the endogenous gene is the CHI gene (tt5 mutant).

24. A reporter system according to claim 1, wherein the expression of transcription factors is altered.

25. A reporter system according to claim 24, wherein the transcription factors contain a Myb domain.

26. A reporter system according to claim 25, wherein the transcription is selected from the group consisting of PAP1 and/or and PAP2.

27. A reporter system according to claim 24, wherein the transcription factors are overexpressed.

28. A reporter system according to claim 27, wherein overexpression is controlled by an inducible promoter.

29. A reporter system according to claim 27, wherein overexpression is controlled by an a constitutive promoter.

30. A reporter system according to claim 27, wherein overexpression is controlled by the 35S promoter.

31. A reporter system according to claim 27, wherein overexpression is controlled by a dual promoter.

32. A reporter system according to claim 1, wherein the outer stimulus is a pollutant.

33. A reporter system according to claim 32, wherein the pollutant is inorganic.

34. A reporter system according to claim 33, wherein the pollutant is a heavy metal.

35. A reporter system according to claim 34, wherein the heavy is selected from the group consisting of Cu, Zn, Cd, Hg, Pb, Co, Cr, Ni, As, Be, Se, Au, and Ag.

36. A reporter system according to claim 32, wherein the pollutant is organic.

37. A reporter system according to claim 36, wherein the organic pollutant is a nitrogen-containing compound.

38. A reporter system according to claim 37, wherein the compound contains NO₂, NO₃, NH₂ or NH₃.

39. A reporter system according to claim 36, wherein the nitrogen-containing compound comprises part of an explosive.

40. A reporter system according to claim 1, wherein the expression of said gene or genes is altered directly by the presence of a pollutant.

41. A reporter system according to claim 1, wherein the expression of said gene or genes is altered indirectly by the presence of a pollutant.

42. A reporter system according to claim 41, wherein the pollutant is converted to a secondary factor in one or more steps and said secondary factor alters expression of said gene(s).

43. A reporter system according to claim 42, wherein the conversion is facilitated by a microbial catabolic enzyme.

44. A reporter system according to claim 42, wherein the microbial enzyme is “TNT reductase”, facilitating the release of NO₂⁻ from TNT.

45. A reporter system according claim 42, wherein the conversion involves a cascade facilitating an amplification of stimulus.

46. A reporter system according to claim 1, wherein the phenotypic trait may be assessed by visual inspection.

47. A reporter system according to claim 46, wherein the phenotypic trait is a colour.

48. A reporter system according to claim 1, wherein the system further comprises a bio-remediation system.

49. A reporter system according to claim 48, wherein the bio-remediation system comprises the breakdown of the pollutant.

50. a reporter system according to claim 49, wherein the bio-remediation system comprises accumulation of the pollutant, and thus fascilitating thereby facilitating its removal.

51. A reporter system according to claim 50, wherein the accumulation is accomplished by the expression an agent selected from the group consisting of heavy metal binding proteins and or metal transport proteins.

52. A reporter system according to claim 51, wherein the bioremediation system comprises a gene is selected from the group consisting of:
Spombe gene encoding phytochelatin-synthetase(gene bank accession Y08414), Athyrium yokoscense AyPCS1 mRNA for phytochelatin synthase (AB057412), Arabidopsis thaliana putative phytochelatin synthase (AY039951), Arabidopsis thaliana phytochelatin synthase (CAD 1, AF135155), Arabidopsis thaliana putative metallothionin-I gene transcription activator (AY04594), Arabidopsis thaliana phytochelatin synthase (PCS 1, AF093753), Arabidopsis thaliana IRT1, and IRT2 metal transporters (U27590 and T04324), Arabidopsis thaliana AtNramp1,2,3, and 4 metal transporter (AF165125, AF141204, AF202539, and AF202540), Brassica juncea mRNA for phytochelatin synthase (pcs1gene AJ278627), Euphorbia esula cDNA similar to phytochelatin synthetase-like protein (BG459096), Lycopersicon esculentum (Tomato crown gal) 1 similar to Arabidopsis thaliana putative phytochelatin synthetase (BGG130981), Typha latifolia phytochelatin synthase (AF308658), Zea mays phytochelatin synthetase-like protein (CISEZmG, AF160475), and Thalaspi caerulescens ZNT1 heavy metal transporter (AF133267).

53. Genetically modified plant, comprising a reporter system according to claim 1.

54. Genetically modified plant according to claim 53, wherein the plant is a monocotyledoneous plant.

55. Genetically modified plant according to claim 53, wherein the plant is a dicotyledoneous plant.

56. Genetically modified plant according to claim 53, wherein the plant is an annual plant.

57. Genetically modified plant according to claim 53, wherein the plant is a biennial plant.

58. Genetically modified plant according to claim 53, wherein the plant is a perennial plant.

59. Genetically modified plant according to claim 53, wherein the plant belongs to the group of Brassicaceae.

60. Genetically modified plant according to claim 59, wherein the plant belongs to the group consisting of the following species: Brassica napus, B. rapa, and B. junceaas, Brassica oleracea, Brassica napus, Brassica rapa, Raphanus sativus, Brassica juncea), Sinapis alba, Armoracia rusticana, Alliaria petiolata, Arabidopsis thaliana, A. griffithiana, A. lasiocarpa, A. petrea, Barbarea vulgaris, Berteroa incana, Brassica juncea, Brassica nigra, Brassica rapa, Bunias orientalis, Camelina alyssum, Camelina microcarpa, Camelina sativa, Capsella bursa-pastoris, Cardaria draba, Cardaria pubescens, Conringia orientalis, Descurainia incana, Descurainia pinnata, Descurainia sophia, Diplotaxis muralis, Diplotaxis tenuifolia, Erucastrum gallicum, Erysimum asperum, Erysimum cheiranthoides, Erysimum hieracifolium, Erysimum inconspicuum, Hesperis matronalis, Lepidium campestre, Lepidium densiflorum, Lepidium perfoliatum, Lepidium virginicum, Nasturtium officinale, Neslia paniculata, Raphanus raphanistrum, Rorippa austriaca, Rorippa sylvestris, Sinapis alba, Sinapis arvensis, Sisymbrium altissimum, Sisymbrium loeselii, Sisymbrium officinale, Thalaspi arvense, and Turritis glabra.

61. A process for detection of an analyte comprising the steps of:
introducing seeds from a genetically modified plant according to claim 53, to a site to be monitored;
monitoring the phenotype of the resulting plants and, as a bioremediation step optionally removing the plants if they accumulate the analyte.

62. A process according to claim 61, wherein the analyte is a pollutant.

63. A process according to claim 62, wherein the pollutant is inorganic.

64. A process according to claim 63, wherein the inorganic pollutant is a heavy metal.

65. A process according to claim 64, wherein the heavy metal is selected from the group consisting of Cu, Zn, Cd, Hg, Pb, Co, Cr, Ni, As, Be, Se, Au, Ag.

66. process according to claim 65, wherein the detected concentration of heavy metal is at least 0.1 mmol per kg soil.

67. A process according to claim 62, wherein the pollutant is organic.

68. A process according to claim 67, wherein the inorganic pollutant is a nitrogen-containing compound.

69. A process according to claim 68, wherein the compound contains NO₂, NO₃, NH₂ or NH₃.

70. A process for detection of soil pollution according to claim 68, wherein the detected concentration of the nitrogen-containing compound is at least 0.1 mmol per kg soil.

71. A process according to claim 61, wherein the bioremediation step reduces the concentration of the analyte with at least 50%.

72-82. (canceled)
